(19) United States
(12) Patent Application Publication
(10) Pub. No.: US 2008/0038729 A1
(43) Pub. Date: Feb. 14, 2008

(54) Method For Monitoring The Progress Of Cancer

(75) Inventors: Gail Petuna Risbridger, East Malvern (AU); Sally Louise Mellor, Sandringham (AU); Emma Margrat Anne Ball, South Yarra (AU); Hong Wang, Mount Waverley (AU); John Stuart Pedersen, Hawthorn (AU); Catriona McLean, Hawthorn (AU)

Correspondence Address: SCULLY, SCOTT, MURPHY & PRESSER, P.C., 400 GARDEN CITY PLAZA, SUITE 300, GARDEN CITY, NY, 11530, US

(73) Assignee: MONASH UNIVERSITY, Victoria (AU)

(21) Appl. No.: 11/578,711

(22) PCT Filed: Apr. 15, 2005
(86) PCT No.: PCT/AU05/00542

§ 371 Date: Jun. 21, 2007

Related U.S. Application Data
(60) Provisional application No. 60/562,715, filed on Apr. 16, 2004.

(51) Int. Cl. C12Q 1/68; G01N 33/574; G01N 33/68; G01N 33/74

(52) U.S. Cl. 435/6; 435/7.23; 436/86; 436/87

(57) ABSTRACT

The present invention relates generally to a method of diagnosing, predicting or monitoring the development or progress of a cancer and, in particular, to a method of diagnosing, predicting or monitoring the development of or progress of prostate cancer in a mammal. The present invention more specifically provides a method for delineating early stage and advanced stage cancers, or predispositions thereto, by screening for changes in the level of two or more of inhibin-α, activin-β_{A}, activin-β_{B}, activin-β_{C}, activin-β_{D}, activin-β_{E} or follistatin expression in a mammal. The present invention further provides a method for diagnosing or monitoring conditions associated with or characterized by the onset of a cancer.

### FIELD OF THE INVENTION

The present invention relates generally to a method of diagnosing, predicting or monitoring the development or progress of a cancer and, in particular, to a method of diagnosing, predicting or monitoring the development or progress of prostate cancer in a mammal. The present invention more specifically provides a method for delineating early stage and advanced stage cancers, or predispositions thereto, by screening for changes in the level of two or more of inhibin-α, activin-β_{A}, activin-β_{B}, activin-β_{C}, activin-β_{D}, activin-β_{E} or follistatin expression in a mammal. The present invention further provides a method for diagnosing or monitoring conditions associated with or characterized by the onset of a cancer.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge.

A neoplasm is an abnormal mass or colony of cells produced by a relatively autonomous new growth of tissue. Most neoplasms arise from the clonal expansion of a single cell that has undergone neoplastic transformation. The transformation of a normal cell to a neoplastic cell can be caused by a chemical, physical, or biological agent (or event) that alters the cell genome. Neoplastic cells are characterized by the loss of some specialized functions and the acquisition of new biological properties, foremost, the property of relatively autonomous growth. They pass on their heritable biological characteristics to progeny cells. Neoplasms may originate in almost any tissue containing cells capable of mitotic division.

The past, present, and future predicted biological behaviour, or clinical course, of a neoplasm is further classified as benign or malignant, a distinction of great importance in diagnosis, treatment, and prognosis. A malignant neoplasm manifests a greater degree of autonomy, is capable of invasion and metastatic spread, may be resistant to treatment, and may cause death. A benign neoplasm, however, exhibits a lesser degree of autonomy, is usually not invasive and does not metastasize.

“Cancer” is a generic term which generally denotes malignant neoplasms. This is a disease which occurs worldwide and is second only to heart disease as the most common cause of death in western countries. The estimated incidence of cancer in the US, for example, is about 1×10⁶ new cases annually. Nearly 80% of all malignant neoplasms arise in 10 anatomical sites, namely: lung, breast, colon and rectum, prostate, lymph nodes, uterus, bladder, pancreas, blood and stomach.

Prostate cancer is a disease that occurs in men mostly over the age of 50. It can occur in younger men but this is rare. Figures suggest that approximately one in four males above the age of 55 will suffer from a prostate disease of some form. The incidence in Australia of prostatic cancer is high and similarly prevalent rates occur in most countries. Globally, prostate cancer now represents the third highest incidence of cancer after lung cancer (due largely to smoking) and stomach cancer. This represents a significant cost to health care systems and decreases the quality of life of men suffering from this disorder.

Further, the incidence of prostate cancer appears to be increasing. This may be partly due to a ‘real’ increased risk, but is certainly related to the increased likelihood of detection, via PSA tests, and the increased number of TURPs operations. A TURP is done when prostate tissue is removed to improve symptoms of a benign prostate condition. However, in doing so, subsequent pathology sometimes indicates the existence of cancer.

Whether there is a real increase in risk or not, the numbers of cases of prostate cancer will rise due to the population at risk—older men—growing with the lengthening of life expectancy.

Although the causes of prostate cancer are not fully understood, men with a family history of prostate cancer in a first degree relative have two to three times the risk of developing the disease, indicating a role for genetic predisposition. However, the majority of prostate cancers are sporadic and unrelated to family history.

Caught early on, prostate cancer is usually a treatable disease. However, about half the men who are diagnosed with prostate cancer are unfortunately diagnosed at a late stage when the disease is less treatable. In this regard, early stage prostate cancer is generally localised to the prostate. Advanced prostate cancer, although having originated in the prostate, has generally spread beyond the prostate to other parts of the body signals a significantly less hopeful prognosis.

Prostate cancer pathologies are graded with a Gleason grading from 1 to 5 in order of increasing malignancy. Cribriform pathological patterns are sometimes observed at the stage of Gleason Grade 3 and 4. Cribriform pathologies are associated with prostate cancer progression and poor patient outcome.

Accordingly, although some tests for diagnosing cancers, such as prostate cancer are currently available, a significant need exists to develop means of both reliable and early detection of cancers and, still more importantly, their classification as early stage cancers versus advanced stage cancers. This provides the patient with the possibility of better tailored treatment regimes and potentially a significantly better prognosis.

In this regard, some advances have been made in the context of identifying changes in levels of inhibin, activin and follistatin protein and gene expression as a marker of the onset of a cancer.

Activins, composed of two β-subunits, β_{A} and/or β_{B}, and their antagonists the inhibins (combinations of an α- and either of the two α-subunits) are members of the transforming growth factor (TGF)-β superfamily [Vale et al. (1990) In Peptide growth factors and their receptors: Handbook of Experimental Physiology, Vol. 95 (Eds, Sporn, M. and Roberts, A.) Springer-Verlag, Berlin, pp. 211-248]. Activins regulate cell growth or differentiation by binding activin receptors and initiating a signaling cascade [Pangas et al. (2000), Trends Endocrinol Metab, 11, 309-314]. Changes in expression of inhibin/activin subunits, activin receptors, or the activin-binding proteins follistatin (FS315 and FS288) have been shown to influence growth of a variety of types of cells. Tumor suppressor activity of inhibin-α in the gonads and adrenals has been recorded in transgenic mice bearing a targeted deletion of the inhibin-α subunit [Matzuk et al. (1992), Nature, 360, 313-9; Matzuk et al. (1994), Semin Cancer Biol, 5, 37-45; Matzuk et al. (1994), Proc Natl Acad Sci USA, 91, 8817-21; Matzuk et al. (1996), Recent Prog Horm Res, 51, 123-54, 1996; Cipriano et al. (2000), Endocrinology, 141, 2319-27; Lopez et al. (1999), Oncogene, 18, 7303-9]. As a tumor suppressor, it was predicted that decreased expression of inhibin-α may confer increased malignant potential. Specifically, it was demonstrated that methylation of the inhibin-α gene was observed more frequently in malignant tissues compared to normal tissues [Schmitt et al. (2002), Mol Endocrinol, 16, 213-20; Balanathan et al., Journal of Molecular Endocrinology, 32, 55-67, 2004].

Inhibin-α and activin-β subunits, and follistatins, are synthesized in the human prostate. Studies on transrectal ultrasound needle biopsies from 15 individuals with benign prostatic hyperplasia (BPH) and 12 patients with cancer, have shown a loss of inhibin-α subunit mRNA by in situ hybridization and loss of protein expression based on immunolocalisation [Mellor et al. (1998), J Clin Endocrinol Metab, 83, 969-975.]. Using an immunopurified sheep polyclonal antibody αC41 against recombinant bovine inhibin-α fusion protein and a polyclonal antibody αN320 against a fusion protein consisting of amino acids 1-26 of the α_{N} region of bovine inhibin-α, staining in the non-malignant epithelium with both of these antibodies has been reported, but no staining in any of the 12 cancers with Gleason score 7-10. In subsequent studies, these observations have been confirmed and loss of inhibin-α subunit expression has been determined to be due to LOH and/or promoter hypermethylation [Schmitt et al., 2002, supra; Balanathan et al., 2004 supra].

Activin β_{A}- and β_{B}-subunits are also localized to the epithelium of benign tissues and poorly differentiated adenocarcinomas of the prostate [Thomas et al. (1997), J Clin Endocrinol Metab, 82, 3851-9; Thomas et al. (1998), Prostate, 34, 34-43]. Follistatin expression is noted in both benign epithelium and poorly differentiated cancer. Antibodies, raised to different isoforms, show distinct labelling patterns in malignant versus benign epithelia suggesting differential production by these prostatic compartments [Thomas 1997 TGFb, supra].

In work leading up to the present invention, it has been surprisingly and unexpectedly found that analysis of the levels of two or more of inhibin-α, activin-β_{A}, activin-β_{B}, activin-β_{C}, activin-β_{D}, activin-β_{E} or follistatin provides a more sensitive and accurate method for detecting and classifying early stage cancers versus advanced stage cancers than analysis of any one of those markers alone. Still further, and in a related aspect, it has also been determined that an increase in the levels of any one of activin-β_{A}, activin-β_{B} or follistatin provides a marker of the onset of an advanced stage cancer. Although the use of these markers in the context of a panel, as detailed above, provides a particularly sensitive and accurate diagnostic test, the findings in respect of the diagnostic value of activin-β_{A}, activin-β_{B} and follistatin, individually, in the context of detecting advanced cancer are nevertheless of value in terms of providing a broad range of diagnostic tools which can be applied in relation to cancer diagnostics.

These findings have now facilitated the development of a means for the highly sensitive detection and accurate classification of the onset of early stage versus advanced stage cancers, in particular early stage versus advanced stage prostate cancer. Also provided are means of monitoring the progress of cancers and means of detecting the existence of a predisposition to developing an early stage or advanced stage cancer. In the context of prostate cancer, the latter is particularly important to enable the identification and design of treatment regimes.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention is directed to a method of detecting the onset of a neoplasm or a predisposition to developing a neoplasm in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage neoplasm or a predisposition thereto.

Another aspect of the present invention provides a method of detecting the onset of a malignant neoplasm of the breast, ovary, thyroid, testis or adrenal gland or a predisposition to developing a malignant neoplasm of the breast, ovary, thyroid, testis or adrenal gland in a mammal said method comprising screening for the level of two or more of inhibin-α, activin-β_{A}, activin-β_{B}, activin-β_{C}, activin-β_{D}, activin-β_{E} or follistatin, protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage malignant neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage malignant neoplasm or a predisposition thereto.

In yet another aspect, the present invention provides a method of detecting the onset of a malignant neoplasm of the oesophagus, stomach, colon, rectum, kidney, bladder, small intestine, large intestine, larynx, nasal cavity, throat, neural tissue or endometrium or a predisposition to developing an advanced malignant neoplasm of the oesophagus, stomach, colon, rectum, kidney, bladder, small intestine, large intestine, larynx, nasal cavity, throat, neural tissue or endometrium in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage malignant neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage malignant neoplasm or a predisposition thereto.

In still another aspect, the present invention provides a method of detecting the onset of a malignant neoplasm of the cervix, brain, skin, lymph node, lung, salivary gland, liver, gallbladder or pancreas or a predisposition to developing an advanced malignant neoplasm of the cervix, brain, skin, lymph node, lung, salivary gland, liver, gallbladder or pancreas in a mammal said method comprising screening for the level of inhibin-α, activin-β_{A}, activin-β_{B}, activin-β_{C}, activin-β_{D}, activin-β_{E} or follistatin protein and/or gene expression in said mammal wherein a decrease in the level of two or more of inhibin-α, activin-β_{A}, activin-β_{B}, activin-β_{C}, activin-β_{D}, activin-β_{E} or follistatin protein and/or gene expression is indicative of the onset of an early stage malignant neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage malignant neoplasm or a predisposition thereto.

A further aspect of the present invention provides a method of detecting the onset of a prostate malignant neoplasm or a predisposition to developing a prostate malignant neoplasm in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage prostate malignant neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage prostate malignant neoplasm or a predisposition thereto.

Another further aspect of the present invention provides a method of detecting the onset of an early stage cancer of the breast, ovary, thyroid, testis or adrenal gland or a predisposition to developing an early stage cancer of the breast, ovary, thyroid, testis or adrenal gland in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of said early stage cancer or a predisposition thereto.

In another aspect, the present invention provides a method of detecting the onset of an early stage cancer of the oesophagus, stomach, colon, rectum, kidney, bladder, small intestine, large intestine, larynx, nasal cavity, throat, neural tissue or endometrium or a predisposition to developing an early stage cancer of oesophagus, stomach, colon, rectum, kidney, bladder, small intestine, large intestine, larynx, nasal cavity, throat, neural tissue or endometrium in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of said early stage cancer or a predisposition thereto.

In yet another aspect, the present invention provides a method of detecting the onset of an early stage cancer of the cervix, brain, skin, lymph note, lung, salivary gland, liver, gallbladder or pancreas or a predisposition to developing an early stage cancer of cervix, brain, skin, lymph note, lung, salivary gland, liver, gallbladder or pancreas in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of said early stage cancer or a predisposition.

In still another aspect the present invention provides a method of detecting the onset of an early stage prostate cancer or a predisposition to developing an early stage prostate cancer in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage prostate cancer or a predisposition thereto.

In yet still another aspect the present invention provides a method of detecting the onset of an advanced stage cancer of the breast, ovary, thyroid, testis or adrenal gland or a predisposition to developing an advanced stage cancer of the breast, ovary, thyroid, testis or adrenal gland in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein an increase in the level of inhibin-α, activin-β_{A}, activin-β_{B}, activin-β_{C}, activin-β_{D}, activin-β_{E} or follistatin protein and/or gene expression is indicative of the onset of said advanced stage cancer or a predisposition thereto.

In still yet another aspect, the present invention provides a method of detecting the onset of an advanced stage cancer of the oesophagus, stomach, colon, rectum, kidney, bladder, small intestine, large intestine, larynx, nasal cavity, throat, neural tissue or endometrium or a predisposition to developing an advanced stage cancer of oesophagus, stomach, colon, rectum, kidney, bladder, small intestine, large intestine, larynx, nasal cavity, throat, neural tissue or endometrium in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein an increase in the level of inhibin-α, activin-β_{A}, activin-β_{B}, activin-β_{C}, activin-β_{D}, activin-β_{E} or follistatin protein and/or gene expression is indicative of the onset of said advanced stage cancer or a predisposition thereto.

In yet another aspect, the present invention provides a method of detecting the onset of an advanced stage cancer of the cervix, brain, skin, lymph note, lung, salivary gland, liver, gallbladder or pancreas or a predisposition to developing an advanced stage cancer of the cervix, brain, skin, lymph note, lung, salivary gland, liver, gallbladder or pancreas in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein an increase in the level of inhibin-α, activin-β_{A}, activin-β_{B}, activin-β_{C}, activin-β_{D}, activin-β_{E} or follistatin protein and/or gene expression is indicative of the onset of said advanced stage cancer or a predisposition thereto.

In a further aspect the present invention provides a method of detecting the onset of an advanced stage prostate cancer or a predisposition to developing an advanced stage prostate cancer in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein an increase in the level of inhibin-α, activin-β_{A}, activin-β_{B}, activin-β_{C}, activin-β_{D}, activin-β_{E} or follistatin protein and/or gene expression is indicative of the onset of an advanced stage prostate cancer or a predisposition thereto.

In yet another further aspect the present invention provides a method of detecting the onset of metastatic prostate cancer or a predisposition to developing metastatic prostate cancer in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein an increase in the level of inhibin-α, activin-β_{A}, activin-β_{B}, activin-β_{C}, activin-β_{D}, activin-β_{E} or follistatin protein and/or gene expression is indicative of the onset of metastatic prostate cancer or a predisposition to developing metastatic prostate cancer.

Another aspect of the present invention is directed to a method of monitoring for the onset or progression of a neoplasm in a mammal said method comprising screening for the modulation in the level of two or more of inhibin-α, activin-β_{A}, activin-β_{B}, activin-β_{C}, activin-β_{D}, activin-β_{E} or follistatin in said mammal wherein the level of said inhibin-α, activin-β_{A}, activin-β_{B}, activin-β_{C}, activin-β_{D}, activin-β_{E} or follistatin relative to the normal level of inhibin is indicative of the onset or progression of said neoplasm.

Still another aspect of the present invention provides a diagnostic kit for assaying biological samples comprising an agent for detecting two or more of inhibin-α, activin-β_{A}, activin-β_{B}, activin-β_{C}, activin-β_{D}, activin-β_{E} or follistatin protein or encoding nucleic acid molecule and reagents useful for facilitating the detection by the agent in the first compartment. Further means may also be included, for example, to receive a biological sample.

In a related aspect all the previously described embodiments of the present invention which related to the detection and/or monitoring of an onset or predisposition to an onset of an advanced stage cancer should extend to analysis based on screening for changes to the levels of any one of activin-β_{A}, activin-β_{B}, and follistatin.

### BRIEF DESCRIPTION OF THE DRAWINGS

FIG. 1 is a graphical representation of inhibin-α, activin-β_{C}, follistatin 315 and activin-β_{A} expression in Gleason grade 4 or 5 prostate cancers. The intensity of immunohistochemical staining for each of the four markers in high grade prostate cancer was compared to benign secretory epithelium within the same tissue section. Intensity was scored as no staining=1, variable+/−staining=2, 1+ staining=3, 2+ staining=4, 3+ staining=5.

Immunostaining for inhibin-α, activin-β_{C} and follistatin 315 significantly increased in high grade cancer compared to benign secretory epithelium. * p=0.01 to 0.05, ** p=0.001 to 0.01, *** p<0.001.

FIG. 2 shows the difference in inhibin-α, activin-β_{C}, follistatin 315 and activin-β_{A} expression in cribriform prostate cancers compared to benign epithelium. The intensity of immunohistochemical staining for inhibin-α, activin-β_{C}, follistatin 315 and activin-β_{A} in prostate tumours with cribriform pathology was compared to adjacent benign secretory epithelium. Intensity was scored as no staining=1, variable+/−staining=2, 1+ staining=3, 2+ staining=4, 3+ staining=5 and plotted on a scatter diagram. The horizontal bar represents the mean of each immunostaining score in either benign epithelium or cribriform cancer.

All four markers were expressed in benign secretory epithelium. Inhibin-α, activin-β_{C} and follistatin 315 immunostaining were significantly increased in cribriform cancers compared to benign epithelium (p<0.05).

FIG. 3 shows the effect on proportion of LNCaP cells in S-phase when activin-β_{C} or inhibin-α is overexpressed. FACS analysis was performed on LNCaP cells transiently transfected with activin-β_{C} cDNA and LNCaP cells stably transfected with inhibin-α cDNA. In both cases, controls consisted of expression vector lacking the cDNA insert.

LNCaP cells overexpressing either activin-β_{C} or inhibin-α (black bars) demonstrated a significant reduction in proportion of cells in S-phase compared to LNCaP cells not transfected with activin-β_{C} or inhibin-α cDNA (p<0.05; grey bars).

FIG. 4 shows the effect on proportion of PC3 cells in S-phase when activin-β_{C} or inhibin-α is overexpressed. FACS analysis was preformed on PC3 cells transiently transfected with activin-β_{C} cDNA and PC3 cells stably transfected with inhibin-α cDNA. In both cases, controls consisted of expression vector lacking the cDNA insert.

PC3 cells overexpressing either activin-β_{C} or inhibin-α (black bars) demonstrated a significant increase in proportion of cells in S-phase compared to PC3 cells not expressing activin-β_{C} or inhibin-α (p<0.05; grey bars).

FIG. 5 shows the detection of expression of activin-β_{C} (and/or activin-β_{E}) [panel A], follistatin 315 [panel B] and activin-β_{A} [panel C] in tissue from patients with prostate cancer metastases to the lymph node. Immunostaining for activin-β_{C}, follistatin and activin-β_{A} was localised to prostate cancer tumour cells with no staining observed in mouse IgG negative control [panel D].

FIG. 6 is a graphical representation of activin-β_{C} and follistatin 315 expression in metastatic prostate cancer compared to benign prostatic secretory epithelium.

Activin-β_{C} and follistatin 315 protein intensity was significantly increased in bone metastases compared to benign prostatic secretory epithelium (p<0.05).

FIG. 7 is a graphical representation of inhibin-α, activin-β_{C}, follistatin 315 and activin-β_{A} expression in Gleason grade 4 or 5 prostate cancers compared to Gleason grade 3 prostate cancer within the same tissue section. Immunostaining intensity was scored as no staining=1, variable+/−staining=2, 1+ staining=3, 2+ staining=4, 3+ staining=5.

Immunostaining for activin-β_{C}, follistatin 315 and activin-β_{A} significantly increased in Gleason grade 4 or 5 prostate cancer compared to Gleason grade 3 prostate cancer. * p=0.01 to 0.05, ** p=0.001 to 0.01, *** p<0.001.

FIG. 8 shows the difference in inhibin-α, activin-β_{C}, follistatin 315 and activin-β_{A} expression in cribriform prostate cancers compared to Gleason grade 3 prostate cancer. The intensity of immunohistochemical staining for inhibin-α, activin-β_{C}, follistatin 315 and activin-β_{A} in prostate tumours with cribriform pathology was compared to adjacent Gleason grade 3 prostate cancer. Intensity was scored as no staining=1, variable+/−staining=2, 1+ staining=3, 2+ staining=4, 3+ staining=5.

Immunostaining for inhibin-α, activin-β_{C} and follistatin 315 significantly increased in cribriform pattern prostate cancer compared to Gleason grade 3 prostate cancers. * p=0.01 to 0.05, ** p=0.001 to 0.01.

FIG. 9 is a graphical representation of the effect on monolayer wound healing when inhibin α is overexpressed in LNCaP cells. Monolayers were wounded by scraping an approximately 1 mm thick line along the middle of the wells. Measurements of the width of the wound were taken over time and expressed as a percentage of wound closure, where time 0 represents 100% wound. The data from inhibin α transfected and empty vector transfected clones are pooled in the graph. The symbols represent: closed grey, parental cell line; open grey, empty vector transfected clones, closed black, inhibin α transfected clones. Overall, LNCaP cells overexpressing inhibin α (black) demonstrated slower rate of wound closure compared the cells not expressing inhibin α.

FIG. 10 is a graphical representation of the effect on subcutaneous tumor growth when inhibin α is overexpressed in LNCaP cells. Male SCID mice were inoculated under the skin with parental LNCaP, three empty vector transfected clones and three inhibin α transfected clones. Tumor rate was determined by tumor volume (V) using the equation V=(L×W²)×0.5 in which V=volume, L=length, and W=width. Values represents mean ±SE(n=15). The bars represent: closed grey, parental cell line; open grey, empty vector transfected clones; closed black, inhibin α transfected clones. ** p 0.001-0.01; *** p<0.001 significant difference between the tumor volumes of empty vector transfected clones (L18 only)/inhibin α transfected clones and parental LNCaP cells. No significant (ns) difference between two of the empty vector transfected clones and the parental LNCaP cells. Overall, LNCaP cells overexpressing inhibin α (black) demonstrated reduction in tumor size compared to the parental LNCaP cells.

FIG. 11 is a graphical representation of the effect on intraprostatic tumor growth when inhibin α is overexpressed in LNCaP cells. Male SCID mice prostates were inoculated with parental LNCaP, three empty vector transfected clones and three inhibin α transfected clones. The prostate tumors formed after orthotopical injections of all the cells. Excised tumors were weighed (mg) to determine the rate of tumor growth. Values represents mean ±SE(n=10). The bars represent: closed grey, parental cell line; open grey, empty vector transfected clones; closed black, inhibin α transfected clones. * p 0.01-0.05; *** p<0.001 significant difference between parental LNCaP cells and the inhibin α overexpressing cells. Ns, no significant difference between parental LNCaP cells and the empty vector transfected clones. Overall, LNCaP cells overexpressing inhibin α (black) demonstrated a decrease in tumor weights compared to the parental LNCaP cells.

### DETAILED DESCRIPTION OF THE INVENTION

The present invention is predicated, in part, on the surprising determination that analysis of the levels of two or more of inhibin-α, activin-β_{A}, activin-β_{B}, activin-β_{C}, activin-β_{D}, activin-β_{E} or follistatin provide a significantly more sensitive and accurate means of detecting and/or assessing a cancer and, even more particularly, determining whether the identified cancer corresponds to an early stage cancer or a late stage/advanced cancer, than any one of these markers alone. Nevertheless, the surprising determination that increased levels of activin-β_{A}, activin-β_{B} or follistatin, alone, are predictive of the onset or predisposition to the onset of an advanced stage cancer are also of value in terms of providing a wide range of diagnostic tools from which to select and apply the detection means most appropriate for a given diagnostic or monitoring situation. These findings have therefore now facilitated the development of a broad range of diagnostic/prognostic tools applicable to cancer detection and, in particular, the development of highly sensitive and informative means of diagnosing and classifying cancers such as prostate cancer.

Accordingly, one aspect of the present invention is directed to a method of detecting the onset of a neoplasm or a predisposition to developing a neoplasm in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage neoplasm or a predisposition thereto.

Preferably, said method is directed to detecting two or more of inhibin-α, activin-β_{A}, activin-β_{C} or follistatin.

Reference to a “neoplasm” should be understood as a reference to an encapsulated or unencapsulated growth of neoplastic cells. Reference to a “neoplastic cell” should be understood as a reference to a cell exhibiting abnormal growth. The term “growth” should be understood in its broadest sense and includes reference to proliferation.

The phrase “abnormal growth” in this context is intended as a reference to cell growth which, relative to normal cell growth, exhibits one or more of an increase in the rate of cell division, an increase in the number of cell divisions, an increase in the length of the period of cell division, an increase in the frequency of periods of cell division or uncontrolled proliferation. Without limiting the present invention in any way, the common medical meaning of the term “neoplasia” refers to “new cell growth” that results as a loss of responsiveness to normal growth controls, eg. to neoplastic cell growth. Neoplasias include “tumours” which may be either benign, pre-malignant or malignant. The term “neoplasm” should be understood as a reference to a lesion, tumour or other encapsulated or unencapsulated mass or other form of growth which comprises neoplastic cells.

The term “neoplasm”, in the context of the present invention should be understood to include reference to all types of cancerous growths or oncogenic processes, metastatic tissues or malignantly transformed cells, tissues or organs irrespective of histopathologic type or state of invasiveness, where the tissue in issue expresses inhibin, activin subunit or follistatin either constitutively or subsequently to an appropriate stimulus.

The neoplastic cells comprising the neoplasm may be any cell type, derived from any tissue, such as an epithelial or non-epithelial cell. Although the present invention is preferably directed to the diagnosis of malignant neoplasms, the diagnosis and/or monitoring of non-malignant neoplasms is not excluded. In a preferred embodiment, the subject neoplasm is a neoplasm of the prostate, ovary, skin, breast, lymph node, lung, salivary gland, liver, gallbladder, pancreas, oesophagus, stomach, colon, rectum, kidney, bladder, endometrium, cervix, adrenal gland, thyroid, brain, small intestine, large intestine, larynx, nasal cavity, throat cancer, neural tumours or testis and even more preferably a malignant neoplasm. Reference to the terms “malignant neoplasm” and “cancer” herein should be understood to be interchangeable.

Accordingly, in one embodiment the present invention provides a method of detecting the onset of a malignant neoplasm of the breast, ovary, thyroid, testis or adrenal gland or a predisposition to developing a malignant neoplasm of the breast, ovary, thyroid, testis or adrenal gland in a mammal said method comprising screening for the level of two or more of inhibin-α, activin-β_{A}, activin-β_{B}, activin-β_{C}, activin-β_{D}, activin-β_{E} or follistatin, protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage malignant neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage malignant neoplasm or a predisposition thereto.

In one preferred embodiment, said neoplasm is a neoplasm of the breast.

In another preferred embodiment said neoplasm is a neoplasm of the ovary.

In another embodiment, the present invention provides a method of detecting the onset of a malignant neoplasm of the oesophagus, stomach, colon, rectum, kidney, bladder, small intestine, large intestine, larynx, nasal cavity, throat, neural tissue or endometrium or a predisposition to developing an advanced malignant neoplasm of the oesophagus, stomach, colon, rectum, kidney, bladder, small intestine, large intestine, larynx, nasal cavity, throat, neural tissue or endometrium in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage malignant neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage malignant neoplasm or a predisposition thereto.

In still another embodiment, the present invention provides a method of detecting the onset of a malignant neoplasm of the cervix, brain, skin, lymph node, lung, salivary gland, liver, gallbladder or pancreas or a predisposition to developing an advanced malignant neoplasm of the cervix, brain, skin, lymph node, lung, salivary gland, liver, gallbladder or pancreas in a mammal said method comprising screening for the level of inhibin-α, activin-β_{A}, activin-β_{B}, activin-β_{C}, activin-β_{D}, activin-β_{E} or follistatin protein and/or gene expression in said mammal wherein a decrease in the level of two or more of inhibin-α, activin-β_{A}, activin-β_{B}, activin-β_{C}, activin-β_{D}, activin-β_{E} or follistatin protein and/or gene expression is indicative of the onset of an early stage malignant neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage malignant neoplasm or a predisposition thereto.

The present invention particularly provides a method of detecting the onset of a prostate malignant neoplasm or a predisposition to developing a prostate malignant neoplasm in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage malignant neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage prostate malignant neoplasm or a predisposition thereto.

In accordance with these aspects, the present invention is preferably directed to detecting two or more of inhibin-α, activin-β_{A}, activin-β_{C} or follistatin.

More preferably, the present invention is directed to detecting any three of inhibin-α, activin-β_{A}, activin-β_{C} or follistatin and still more preferably, all four of inhibin-α, activin-β_{A}, activin-β_{C} or follistatin.

Without limiting the present invention to any one theory or mode of action, it has been determined that the levels of each of inhibin-α, activin-β_{A}, activin-β_{B}, activin-β_{C}, activin-β_{D}, activin-β_{E} and follistatin, and in particular inhibin-α, activin-β_{A}, activin-β_{C} and follistatin, are linked to the regulation of cellular proliferation, in the context of a switching mechanism. Specifically, cancers are generally multi-step processes involving an initial transition from non-malignant to malignant status and, following a shift to pre-malignant lesions and localized cancer, the shift to metastasis. In the non-malignant state the activities of tumor suppressor molecules are thought to dominate. It has now been found that a loss in the levels of one or more of inhibin-α, activin-β_{A}, activin-β_{B}, activin-β_{C}, activin-β_{D}, activin-β_{E} and follistatin is linked to the development of an early stage cancer. If left untreated, some of these early stage cancers will progress to highly aggressive, metastatic forms while others will not.

Since it cannot be predicted, at this early stage, how any given cancer is likely to progress, there is significant value and importance in enabling the routine, accurate and sensitive identification of the onset or predisposition to the onset of an early stage cancer. In general, early stage cancers which are destined to progress will shift to a moderate grade, these being a class of neoplasms some of which will continue to progress to an advanced stage (and likely poor outcome), and some of which will not. To date, the prognostic assessment of moderate grade prostate cancers have proved extremely difficult an unreliable. It should therefore be understood that the detection method of the present invention does not extend to classifying or otherwise assessing neoplastic prostate cells of this moderate grade or conditions characterised by neoplastic prostate cells at this differentiative stage.

As disease progression continues, oncogenic activities are thought to prevail and there is a switch in expression levels of inhibin-α, activin-β_{A}, activin-β_{B}, activin-β_{C}, activin-β_{D}, activin-β_{E} and follistatin indicating a shift to an advanced disease status and metastasis.

Reference to the subject neoplasm being an “advanced stage” neoplasm or cancer should be understood as a reference to a high grade cancer, which high grade cancer may or may not have metastasised or otherwise spread beyond the organ or tissue in which it has originated. Without limiting the invention to any one theory or mode of action, metastases can generally form via distribution of the neoplastic cells through the bloodstream or the lymphatic channels or across body cavities such as the pleural or peritoneal spaces, thus setting up secondary tumours at sites distant from the original tumours. Any individual primary tumour will exhibit its own pattern of local behaviour and metastases. It should also be understood that reference to an “advanced stage” neoplasm or cancer encompasses any level or degree of spreading of the neoplastic cells beyond the organ or tissue in which it originated, whether that be relatively localised spreading in the immediate vicinity of the organ or tissue in issue or the more significant spreading of the neoplastic cells to other regions of the body, which accords with the more commonly understood notion of “metastatic” cancer. It also encompasses the form of cancer which is characterised by the development of metastases subsequently to removal of the organ or tissue in which the cancer originated. Accordingly, for example, “advanced” prostate cancer may develop, or a predisposition to development may be found to exist, subsequently to removal of the prostate.

Reference to an “early stage” neoplasm or cancer is a reference to a low grade, non-metastatic neoplasm.

The method of the present invention is preferably directed to detecting and classifying early stage versus advanced stage prostate cancers. In this regard, reference to a neoplasm “grade” (as it applies to the definition of “early stage” and “late stage”) in the context of prostate cancer should be understood as a reference to the classification of the neoplasm in accordance with the Gleason grading system. Without limiting the present invention to any one theory or mode of action, the Gleason system is based on the architectural pattern of the glands of the prostate tumour. It evaluates how effectively the cells of any particular cancer are able to structure themselves into glands resembling those of the normal prostate. The ability of a tumour to mimic normal gland architecture is termed its differentiation, and, in general, a tumour whose structure is nearly normal (well differentiated) generally exhibits biological behaviour closer to normal, that is, is not aggressively malignant.

The Gleason grading from very well differentiated (grade 1) to very poorly differentiated (grade 5) is usually assessed as follows:

(i) Gleason Grades 1 and 2

These two grades generally resemble normal prostate. Both of these grades are composed by mass; in grade 2 they are more loosely aggregated, and some glands invade the surrounding stroma.
   (ii) Gleason Grade 3
This is the most common grade observed in patients and is also considered well differentiated (like grades 1 and 2). This is due to all three grades exhibiting a normal “gland unit” like that of a normal prostate; that is, every cell is part of a circular row which forms the lining of the lumen. The lumen comprises prostatic secretion like normal prostate, and each gland unit is surrounded by stroma which keeps the gland units apart. In contrast to grade 2, wandering of invasion into the stroma is prominent and is the main defining feature.
   (iii) Gleason Grade 4
Where significant levels of Gleason grade 4 are present, patient prognosis is usually significantly worsened. This grade is characterised by a significant loss of architecture, specifically loss of the normal gland unit. In fact, grade 4 is generally identified by loss of the ability to form individual, separate gland units, each with its separate lumen.
   (iv) Gleason Grade 5
Gleason grade 5 usually predicts another significant step towards poor prognosis. This grade is also characterised by lack of evidence of any gland unit formation. Grade 5 is generally termed “undifferentiated”, due to its features not being significantly distinguishing from undifferentiated cancers which occur in other organs.

A Tabulated Representation of the Gleason Grading System is Provided in Table 1.

TABLE 1
Gleason grading system for prostatic adenocarcinoma: histological patterns
(Bostwick & Dundore, Biopsy Pathology of the Prostrate, Chapman & Hall Medical, UK)

| | | | | Size | | |
|---|---|---|---|---|---|---|
| Pattern | Peripheral border | Infiltrative appearance | Appearance of glands | of glands | Architecture of glands | Cytoplasm |
| 1 | Circumscribed, | Minimal | Simple, round, monotonously | Medium, | Closely-packed | Similar to benign |
| | pushing, | | replicated | regular | rounded masses | epithelium |
| | expansile | | | | | |
| 2 | Less circumscribed; | Mild, with definite | Simple, round, some | Medium, | Loosely-packed | Similar to benign |
| | early infiltration | separation of glands | variability in shape | less regular | rounded masses | epithelium |
| 3A | Infiltration | Marked | Angular, with variation in | Medium to | Variable packed | More basophilic than |
| | | | shape | large | irregular masses | patterns 1 and 2 |
| 3B | Infiltration | Marked | Angular, with variation in | Small | Variable packed | More basophilic than |
| | | | shape | | irregular masses | patterns 1 and 2 |
| 3C | Smooth, rounded | Marked | Papillary and cribriform | Irregular | Round to elongate | More basophilic than |
| | | | | | masses | patterns 1 and 2 |
| 4A | Ragged infiltration | Marked | Microacinary, papillary, | Irregular | Fused, with chains and | Dark |
| | | | cribriform | | and cords | |
| 4B | Ragged infiltration | Marked | Microacinary, papillary, and | Irregular | Fused, with chains | Clear (“hypernephroid”) |
| | | | cribriform | | and cords | |
| 5A | Smooth, rounded | Marked | Comedocarcinoma | Irregular | Round to elongate | Variable |
| | | | | | masses | |
| 5B | Ragged infiltration | Marked | Difficult to identify gland | Fused sheets | | Variable |
| | | | lumens | and masses | | |

In addition to the fundamental grading system, each patient is also given a Gleason score. The Gleason score is based on the summation of the grades of the two most common architectural patterns in a tissue sample. This provides a slightly more refined means of classifying the neoplasm of a given patient. For example, the lowest possible Gleason score is 2 (1+1), where both the primary and secondary patterns exhibit a Gleason grade of 1. Very typical Gleason scores might be 5 (2+3), where the primary pattern has a Gleason grade of 2 and the secondary patterns has a grade of 3, or 6 (3+3), a pure pattern. Another typical Gleason score might be 7 (4+3), where the primary pattern has a Gleason grade of 4 and the secondary pattern has a grade of 3. Finally, the highest possible Gleason score is 10 (5+5), when the primary and secondary patterns both have the most disordered Gleason grades of 5.

The method of the present invention provides a highly sensitive or accurate means of detecting the onset or predisposition to the onset of a neoplastic condition and, in particular, the delineation of early stage cancers and advanced stage cancers. In the context of prostate cancer and the Gleason grading system, early stage prostate cancer should be understood to encompass a neoplasm comprising cells of Grade 1 or 2 or equivalent grade thereof, while advanced stage prostate cancer should be understood to encompass a neoplasm comprising cells of Gleason grade 4 or 5, or equivalent grade thereof. It should also be understood that although any given prostate cancer may comprise cells of various grades, the present invention is directed to screening those cells which fall within the ambit of “early stage” or “advanced stage” cancer, irrespective of what the overall Gleason score may suggest about the classification of a prostate cancer. For example, a prostate cancer comprising Gleason grade 4 and 2 cells correlates to a Gleason score of 6, this being a score which is equated with a “moderate” grade cancer. However, since the present invention can be designed to analyse subgroups of cells if necessary, for example via analysis of tissue sections, for the purpose of the present invention it is not the overall grade of the neoplasm which is of relevance but the grade of the cells which are the subject of enquiry. For example, the present invention may be directed to analysing the grade 4 component of a neoplasm. The advanced stage neoplasms as defined herein should be understood to correlate to neoplasms which are also routinely termed “advanced cancers”, “aggressive cancers” and “metastatic cancers”, although not all advanced cancers are necessarily metastatic cancers. It should also be understood that reference to an “advanced” cancer, in the context of prostate cancer, encompasses any level or degree of spreading of the neoplastic cells beyond the prostate, whether that be relatively localised spreading in the immediate vicinity of the prostate or the more significant spreading of the neoplastic cells to other regions of the body, which accords with the more commonly understood notion of “metastatic” cancer. As detailed hereinbefore, it also encompasses the form of cancer which is characterised by the development of prostate derived metastases subsequently to removal of the prostate. Accordingly, “advanced” prostate cancer may develop subsequently to removal of the prostate.

Accordingly, the present invention provides a method of detecting the onset of an early stage cancer of the breast, ovary, thyroid, testis or adrenal gland or a predisposition to developing an early stage cancer of the breast, ovary, thyroid, testis or adrenal gland in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of said early stage cancer or a predisposition thereto.

In one preferred embodiment, said neoplasm is a neoplasm of the breast.

In another preferred embodiment said neoplasm is a neoplasm of the ovary.

In still another embodiment, the present invention provides a method of detecting the onset of an early stage cancer of the oesophagus, stomach, colon, rectum, kidney, bladder, small intestine, large intestine, larynx, nasal cavity, throat, neural tissue or endometrium or a predisposition to developing an early stage cancer of oesophagus, stomach, colon, rectum, kidney, bladder, small intestine, large intestine, larynx, nasal cavity, throat, neural tissue or endometrium in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of said early stage cancer or a predisposition thereto.

In yet another preferred embodiment, the present invention provides a method of detecting the onset of an early stage cancer of the cervix, brain, skin, lymph note, lung, salivary gland, liver, gallbladder or pancreas or a predisposition to developing an early stage cancer of cervix, brain, skin, lymph note, lung, salivary gland, liver, gallbladder or pancreas in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of said early stage cancer or a predisposition.

Most preferably, the present invention provides a method of detecting the onset of an early stage prostate cancer or a predisposition to developing an early stage prostate cancer in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage prostate cancer or a predisposition thereto.

In accordance with these preferred embodiments, said markers are preferably inhibin-α, activin-β_{A}, activin-β_{C} or follistatin and said screening is directed to any 3 or all 4 of these markers.

In another aspect the present invention provides a method of detecting the onset of an advanced stage cancer of the breast, ovary, thyroid, testis or adrenal gland or a predisposition to developing an advanced stage cancer of the breast, ovary, thyroid, testis or adrenal gland in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein an increase in the level of inhibin-α, activin-β_{A}, activin-β_{B}, activin-β_{C}, activin-β_{D}, activin-β_{E} or follistatin protein and/or gene expression is indicative of the onset of said advanced stage cancer or a predisposition thereto.

In one preferred embodiment, said neoplasm is a neoplasm of the breast.

In another preferred embodiment said neoplasm is a neoplasm of the ovary.

Most preferably, said high grade cancer is metastatic cancer.

In another embodiment, the present invention provides a method of detecting the onset of an advanced stage cancer of the oesophagus, stomach, colon, rectum, kidney, bladder, small intestine, large intestine, larynx, nasal cavity, throat, neural tissue or endometrium or a predisposition to developing an advanced stage cancer of oesophagus, stomach, colon, rectum, kidney, bladder, small intestine, large intestine, larynx, nasal cavity, throat, neural tissue or endometrium in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein an increase in the level of inhibin-α, activin-β_{A}, activin-β_{B}, activin-β_{C}, activin-β_{D}, activin-β_{E} or follistatin protein and/or gene expression is indicative of the onset of said advanced stage cancer or a predisposition thereto.

Most preferably, said high grade cancer is metastatic cancer.

In yet another preferred embodiment, the present invention provides a method of detecting the onset of an advanced stage cancer of the cervix, brain, skin, lymph note, lung, salivary gland, liver, gallbladder or pancreas or a predisposition to developing an advanced stage cancer of the cervix, brain, skin, lymph note, lung, salivary gland, liver, gallbladder or pancreas in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein an increase in the level of inhibin-α, activin-β_{A}, activin-β_{B}, activin-β_{C}, activin-β_{D}, activin-β_{E} or follistatin protein and/or gene expression is indicative of the onset of said advanced stage cancer or a predisposition thereto.

Most preferably, said high grade cancer is metastatic cancer.

Most preferably, the present invention provides a method of detecting the onset of an advanced stage prostate cancer or a predisposition to developing an advanced stage prostate cancer in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein an increase in the level of inhibin-α, activin-β_{A}, activin-β_{B}, activin-β_{C}, activin-β_{D}, activin-β_{E} or follistatin protein and/or gene expression is indicative of the onset of an advanced stage prostate cancer or a predisposition thereto.

Most preferably, said high grade prostate cancer is metastatic prostate cancer.

According to this preferred embodiment, the present invention provides a method of detecting the onset of metastatic prostate cancer or a predisposition to developing metastatic prostate cancer in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein an increase in the level of inhibin-α, activin-β_{A}, activin-β_{B}, activin-β_{C}, activin-β_{D}, activin-β_{E} or follistatin protein and/or gene expression is indicative of the onset of metastatic prostate cancer or a predisposition to developing metastatic prostate cancer.

In accordance with these preferred embodiments, said markers are preferably inhibin-α, activin-β_{A}, activin-β_{C} or follistatin and said screening is directed to any 3 or all 4 of these markers.

The present invention is predicated on the determination that changes in the level of expression of two or more of inhibin-α, activin-β_{A}, activin-β_{B}, activin-β_{C}, activin-β_{D}, activin-β_{E} or follistatin, are indicative of the development of a cancer, in particular low grade or high grade prostate cancer.

Reference to “activin β_{C}” should be understood as a reference to all forms of activin β_{C} and to fragments, derivatives, mutants or variants thereof. “Activin β_{C}” is also interchangeably referred to as “activin β_{C} subunit”. It should also be understood to include reference to any isoforms which may arise from alternative splicing of activin β_{C} mRNA or mutant or polymorphic forms of activin β_{C}. Reference to “activin β_{C}” is not intended to be limiting and should be read as including reference to all forms of activin β_{C} including any protein encoded by the activin β_{C} subunit gene, any subunit polypeptide such as precursor forms which may be generated, and any activin β_{C} protein, whether existing as a monomer, multimer or fusion protein. Multimeric protein forms of activin β_{C} include for example the homodimeric activin C (β_{C}-β_{D}) or the heterodimeric activin AC (β_{A}-β_{C}), activin BC (β_{B}-β_{C}), activin CD (β_{C}-β_{D}) or activin CE (β_{C}-β_{E}) proteins. Accordingly, it should be understood that one may screen for activin β_{C} in its monomeric, homodimeric or heterodimeric form. A corresponding definition applies with respect to “activin-β_{A}, activin-β_{B}, activin-β_{C}, activin-β_{D}, and activin-β_{E}”.

Without limiting the present invention to any one theory or mode of action the structure of activins are similar to one another and other members of the TGFβ superfamily and are based on the conservation of the number and spacing of the cysteines within each subunit and the disulphide linkages between the two subunits that form characteristic cysteine knots. Other similarities relate to dimer formation, the location of the bioactive peptide in the carboxy terminal region of the precursor activin subunit molecule and similar intracellular signalling mechanisms. Human activin β_{C}, for example, in comparison with other TGF-β superfamily members, reveals a typical structure with 9 conserved cysteines and a large precursor molecule that contain a core of hydrophobic amino acids at the N terminus thought to be the secretion signal sequence (Hotten G et al, 1995, Biochem Biophys Res Commun, 206: 608-13). The mouse activin β_{C} also contains 9 conserved cysteines and N terminal hydrophobic amino acids that may serve as a signal peptide (Schmitt et al. 1996, Genomics, 32: 358-66).

Reference to “inhibin α” should be understood as a reference to all forms of inhibin α and to fragments, derivatives, mutants or variants thereof. Inhibin α is also interchangeably referred to as “inhibin α subunit”. It should also be understood to include reference to any isoforms which may arise from alternative splicing of inhibin α mRNA or mutant or polymorphic forms of inhibin α. Reference to “inhibin α” is not intended to be limiting and should be read as including reference to all forms of inhibin α including any protein encoded by the inhibin α subunit gene, any subunit polypeptide, the precursor polypeptide forms pre, pro αN and αC, and any inhibin α protein, whether existing as a monomer, multimer or fusion protein. Multimeric protein forms of inhibin a include for example the heterodimeric αβ polypeptide (for example αβ_{A}, αβ_{B}, αβ_{C}, αβ_{D}, αβ_{B} and αβ_{E}) and the dimeric precursor αC-β polypeptide. Without limiting the present invention in any way, the αN and/or αC regions of precursor α-subunit proteins are found to exist either as part of an existing precursor α-subunit protein or in isolation, for example, following cleavage of said region from a precursor α-subunit protein. Precursor α-subunit proteins exist in many forms including, but not limited to, the forms pre- pro-αN -αC and pro-αC. According to this embodiment of the present invention, detection of α-inhibin proteins, including precursor α-subunit proteins, includes the detection of the αN and/or αC regions both in isolation, and as part of one or more of the various forms of precursor α-subunit protein.

The inhibin-α proteins which are detectable in the tissues from patients, in particular the prostate from patients diagnosed with benign prostate hyperplasia or in the non-malignant regions of prostate may comprise for example, the αN and/or αC regions. The present invention is exemplified, but not limited in any way, by reference to detection of inhibin-α levels via the detection of the αC regions of the inhibin-α protein. Inhibin-α proteins comprising αN and/or αC regions are also referred to as precursor α-subunit proteins. The αN and/or αC regions of precursor α-subunit proteins are found to exist either as part of an existing precursor α-subunit protein or in isolation, for example, following cleavage of said region from a precursor α-subunit protein. Precursor α-subunit proteins exist in many forms including, but not limited to, the forms pre- pro-αN -αC and pro-αC. According to this embodiment of the present invention, detection of inhibin-α proteins, including precursor α-subunit proteins, includes the detection of the αN and/or αC regions both in isolation, and as part of one or more of the various forms of precursor α-subunit protein.

Without limiting the present invention to any one theory or mode of action, it is thought that inhibin-α may undergo different forms of processing and/or cleavage at the α-C region of the inhibin-α subunit. This has been evidenced by the fact that the commonly used diagnostic antibody Groome R1 [Robertson et al., 2001, Mol Cell Endo. 180: 79-86], which is directed to the inhibin-α subunit amino acids 3-24 of the α-C region is unable to detect the presence of the form of inhibin-α which is increased in prostate biopsy samples exhibiting the onset of advanced cancer whereas the monoclonal antibody PO#12 [Robertson et al., 2001, supra], directed to inhibin-α amino acids 73-96 of the α-C region, did detect these increased levels of inhibin-α.

Accordingly, in a preferred embodiment the form of inhibin-α which is detected is the form of inhibin-α which comprises amino acids 73-96 of the α-C region.

Most preferably, said inhibin-α protein is detected utilising the PO#12 monoclonal antibody and said advanced cancer is metastatic cancer.

Reference to “follistatin” should be read as including reference to all forms of follistatin and to fragments, derivatives, mutants or variants thereof including, by way of example, the three protein cores and six molecular weight forms which have been identified as arising from the alternatively spliced mRNAs FS315 and FS288. Accordingly, it should also be understood to include reference to any isoforms which may arise from alternative splicing of follistatin mRNA or mutant or polymorphic forms of follistatin. It should still further be understood to extend to any protein encoded by the follistatin gene, any subunit polypeptide, such as precursor forms which may be generated, and any follistatin protein, whether existing as a monomer, multimer or fusion protein.

The term “mammal” as used herein includes humans, primates, livestock animals (eg. horses, cattle, sheep, pigs, donkeys), laboratory test animals (eg. mice, rats, guinea pigs), companion animals (eg. dogs, cats) and captive wild animals (eg. kangaroos, deer, foxes). Preferably, the mammal is a human or a laboratory test animal. Even more preferably, the mammal is a human.

The present invention is predicated on the finding that levels of two or more of inhibin-α, activin-β_{A}, activin-β_{B}, activin-β_{C}, activin-β_{D}, activin-β_{E} or follistatin, and in particular inhibin-α, activin-β_{A}, activin-β_{C} or follistatin, are modulated in neoplastic tissue as compared to normal tissue or non-malignant neoplastic tissue. In this regard, the person of skill in the art will understand that one may screen for changes to levels at either the protein or the encoding nucleic acid molecule level. To the extent that it is not always specified, reference herein to screening for the level of two or more of “inhibin-α, activin-β_{A}, activin-β_{B}, activin-β_{C}, activin-β_{D}, activin-β_{E} or follistatin” should be understood to include reference to screening for either the protein or its encoding primary RNA transcript or mRNA. Accordingly, it should be understood that the present invention is directed to the correlation of the level of these molecules relative to control levels of these molecules. “Control” levels may be either “normal” or benign levels or the levels in neoplastic cells of a corresponding grade. The “normal” level is the level of protein or encoding nucleic acid molecule, in a biological sample corresponding to the sample being analysed, of an individual who has not developed a neoplasm nor is predisposed to developing a neoplasm. The “normal” level also includes reference to the level of these molecules in non-neoplastic regions of the tissue which is the subject of analysis. This latter method of analysis is a relative form of analysis in terms of the normal and test levels being determined from non-neoplastic and test tissues, respectively, derived from a single individual. However, the method of the present invention should also be understood to encompass non-relative analysis means such as the analysis of test results relative to a standard result which reflects individual or collective results obtained from healthy individuals, other than the patient in issue. Said “normal level” may be a discrete level or a range of levels. In this regard, it should be understood that levels may be assessed or monitored by either quantitative or qualitative readouts. The reference level may also vary between individual forms (such as differently processed forms) of these molecules. Reference to the marker levels of “corresponding grade” neoplastic cells should be understood as a reference to the levels which one observes or detects in any other neoplastic cell of the same grade as the cell which is under analysis, whether that be a cell or cells which are present in the tissue which is the subject of analysis or cells which are found in a corresponding but separate biological sample harvested from either the same individual or a different individual. It should be understood that this form of analysis may be relevant due to the fact that not all cells of a defined grade will necessarily progress in the same manner. For example, not all grade 4 cells may necessarily progress to metastises. Accordingly, differences between similar populations of cells in terms of the levels of the panel of markers defined herein may provide extremely valuable information. One may also seek to compare levels of these markers in cells of different grades. It should also be understood that the discussion, above, in relation to relative versus non-relative analyses, standard results and discrete versus ranges of levels applies equally in this context.

Accordingly, the terms “increase”, “decrease” and “modulation” refer to increases and decreases in inhibin-α, activin-β_{A}, activin-β_{B}, activin-β_{C}, activin-β_{D}, activin-β_{E} and follistatin levels relative either to a control level (or control level range) or to an earlier result determined from the patient in issue, this latter reference point being particularly relevant in the context of the ongoing monitoring of a patient, as hereinafter described.

Without limiting the present invention to any one theory or mode of action, it is proposed that inhibin-α, activin-β_{A}, activin-β_{B}, activin-β_{C}, activin-β_{D}, activin-β_{E} and/or follistatin exhibit dual roles as an indicator of carcinogenesis in the context of the development of both low grade and high neoplasms.

Reference to the “onset” of an advanced stage cancer, in particular an advanced stage prostate cancer, should be understood as a reference to one or more cells of that individual exhibiting an advanced stage growth characteristic. In this regard, the advanced stage cancer may be well developed in that a mass of proliferating cells has developed. Alternatively, the advanced stage cancer may be at a very early stage in that only relatively few divisions of the cells characterising the cancer have occurred at the time of diagnosis. Nevertheless, the method of the present invention facilitates the identification of increased expression of two or more of inhibin-α, activin-β_{A}, activin-β_{B}, activin-β_{C}, activin-β_{D}, activin-β_{E} or follistatin in these cells and, therefore, their detection. As detailed hereinbefore, it has also been determined that an increase in expression of two or more of inhibin-α, activin-β_{A}, activin-β_{B}, activin-β_{C}, activin-β_{D}, activin-β_{E} or follistatin in advanced stage neoplastic tissue can correlate to the future development of very aggressive neoplastic conditions such as metastatic cancer, that is, before evidence of metastases formation occurs. Accordingly, the present invention also extends to the assessment of an individual's predisposition to the development of certain classes of high grade neoplasm, such as metastatic cancer. It should be understood that a corresponding definition applies with respect to the onset or predisposition to the onset of an early stage cancer.

Although the preferred method is to detect an increase in two or more of inhibin-α, activin-β_{A}, activin-β_{B}, activin-β_{C}, activin-β_{D}, activin-β_{E} or follistatin levels in order to diagnose the onset of or predisposition to the onset of an advanced stage cancer, or a decrease in two or more of inhibin-α, activin-β_{A}, activin-β_{B}, activin-β_{C}, activin-β_{D}, activin-β_{E} or follistatin to diagnose the onset or a predisposition to the onset of an early stage cancer, the detection of decreases in inhibin-α, activin-β_{A}, activin-β_{B}, activin-β_{C}, activin-β_{D}, activin-β_{E} or follistatin levels in the context of advanced stage cancers or increase in these molecules in the context of early stage cancers may be desired under certain circumstances. For example, in the context of advanced stage prostate cancer, where a radical prostatectomy is not performed one may seek to monitor for improvements in the disease state of the prostate (characterised by a decrease in marker levels) during the course of prophylactic or therapeutic treatment of the patient. In another example, patients presenting with early stage prostate cancer in the form of very early symptoms of prostate disease or a genetic or environmental predisposition to the development of prostate disease, one may monitor for elevation of low levels of the marker molecules back to normal levels during the course of treatment. In another example, to the extent that the prostate has been removed and analysis of the prostate have revealed high levels of marker and therefore a predisposition to the development of metastatic prostate cancer, one may seek to monitor systemic or appropriately selected localised levels of inhibin-α, activin-β_{A}, activin-β_{B}, activin-β_{C}, activin-β_{D}, activin-β_{E} or follistatin as an indication of either the development or regression of metastases. This aspect of the present invention therefore enables one to monitor the progression of an advanced cancer or predisposition thereto. Similarly, screening for increases in levels of two or more of inhibin-α, activin-β_{A}, activin-β_{B}, activin-β_{C}, activin-β_{D}, activin-β_{E} or follistatin in the context of early stage cancers, in general, provides a means of detecting normalisation of tumour suppression function in the tissue in issue. This may be indicative of an effective treatment regime. It should be understood that in accordance with this aspect of the present invention, inhibin-α, activin-β_{A}, activin-β_{B}, activin-β_{C}, activin-β_{D}, activin-β_{E} or follistatin levels will likely be assessed relative to one or more previously obtained results, as hereinbefore described.

The method of the present invention is therefore useful as a one off test or as an on-going monitor of those individuals thought to be at risk of early stage or advanced stage cancer development or as a monitor of the effectiveness of therapeutic or prophylactic treatment regimes directed to inhibiting or otherwise slowing cancer development. In these situations, mapping the modulation of two or more of inhibin-α, activin-β_{A}, activin-β_{B}, activin-β_{C}, activin-β_{D}, activin-β_{E} or follistatin in any one or more classes of biological samples is a highly sensitive and accurate indicator of the status of an individual or the effectiveness of a therapeutic or prophylactic regime which is currently in use. The method of the present invention should therefore be understood to extend to monitoring for increases or decreases in two or more of inhibin-α, activin-β_{A}, activin-β_{B}, activin-β_{C}, activin-β_{D}, activin-β_{E} or follistatin levels in an individual relative to a control level (as hereinbefore defined) or relative to one or more earlier levels determined from said individual.

Accordingly, another aspect of the present invention is directed to a method of monitoring for the onset or progression of a neoplasm in a mammal said method comprising screening for the modulation in the level of two or more of inhibin-α, activin-β_{A}, activin-β_{B}, activin-β_{C}, activin-β_{D}, activin-β_{E} or follistatin in said mammal wherein the level of said inhibin-α, activin-β_{A}, activin-β_{B}, activin-β_{C}, activin-β_{D}, activin-β_{E} or follistatin relative to the normal level of inhibin is indicative of the onset or progression of said neoplasm.

Preferably said markers are three or four of inhibin-α, activin-β_{A}, activin-β_{C} or follistatin.

In another preferred embodiment, said neoplasm is a malignant breast neoplasm.

In yet another preferred embodiment, said neoplasm is a malignant ovarian neoplasm.

In still another preferred embodiment, said neoplasm is a malignant neoplasm of the thyroid, testis or adrenal gland.

In still another preferred embodiment, said neoplasm is a malignant neoplasm of the prostate, skin, lymph node, lung, salivary gland, liver, gall bladder, pancreas, oesophagus, stomach, colon, rectum, kidney, bladder, small intestine, large intestine, larynx, nasal cavity, throat, neural tissue or endometrium or testis.

In yet another preferred embodiment, said neoplasm is a malignant neoplasm of the prostate.

The method of the present invention has widespread applications including, but not limited to, the diagnostic or prognostic analysis of cancer, in particular prostate cancer, or any condition characterised by the presence of cancer, for example, the conditions associated with advanced prostate cancer such as urine retention, haematuria, urinary incontinence, kidney failure, bone pain, bone fragility, spinal cord damage, osteoarthritis, lethargy, loss of appetite, nausea, diarrhea, constipation or cachexia.

Means of screening for changes in inhibin-α, activin-β_{A}, activin-β_{B}, activin-β_{C}, activin-β_{D}, activin-β_{E} or follistatin (herein referred to as “the markers”) levels in an individual, or biological sample derived therefrom, can be achieved by any suitable method, which would be well known to the person of skill in the art, such as but not limited to:
(i) In vivo detection of the markers. Molecular Imaging may be used following administration of imaging probes or reagents capable of disclosing altered expression levels of the inhibin-α mRNA or protein expression product in the prostate tissues.
   Molecular imaging [Moore et al., BBA, 1402: 239-249, 1988; Weissleder et al. Nature Medicine, 6: 351-355, 2000] is the in vivo imaging of molecular expression that correlates with the macro-features currently visualized using “classical” diagnostic imaging techniques such as X-Ray, computed tomography (CT), MRI, Positron Emission Tomography (PET) or endoscopy. Historically, detection of malignant tumor cells in a background of normal or hyperplastic benign tissue is often based on differences in physical properties between tissues, which are frequently minimal, resulting in low contrast resolution. Application of expression profiling will define the differences in “molecular properties” between cancer and normal tissues that arise as a result of malignant transformation.
(ii) Detection of up-regulation of mRNA expression in the cells by Fluorescent In Situ Hybridization (FISH), or in extracts from the cells by technologies such as Quantitative Reverse Transcriptase Polymerase Chain Reaction (QRTPCR) or Flow cytometric qualification of competitive RT-PCR products [Wedemeyei et al., W. Clinical Chemistry 48: 9 1398-1405, 2002] or array technologies.
   For example, a labelled polynucleotide encoding the markers may be utilized as a probe in a Northern blot of an RNA extract obtained from a tissue. Preferably, a nucleic acid extract from the animal is utilized in concert with oligonucleotide primers corresponding to sense and antisense sequences of a polynucleotide encoding inhibin-α, or flanking sequences thereof, in a nucleic acid amplification reaction such as RT PCR, real time PCR or SAGE. A variety of automated solid-phase detection techniques are also appropriate. For example, a very large scale immobilized primer arrays (VLSIPS™) are used for the detection of nucleic acids as, for example, described by Fodor et al., 1991 and Kazal et al., 1996. The above genetic techniques are well known to persons skilled in the art.
   For example, to detect inhibin-α encoding RNA transcripts, RNA is isolated from a cellular sample suspected of containing the marker RNA, e.g. total RNA isolated from human prostate cancer tissue. RNA can be isolated by methods known in the art, e.g. using TRIZOL™ reagent (GIBCO-BRL/Life Technologies, Gaithersburg, Md.). Oligo-dT, or random-sequence oligonucleotides, as well as sequence-specific oligonucleotides can be employed as a primer in a reverse transcriptase reaction to prepare first-strand cDNAs from the isolated RNA. Resultant first-strand cDNAs are then amplified with sequence-specific oligonucleotides in PCR reactions to yield an amplified product.
   “Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers. These primers will be identical or similar in sequence to opposite strands of the template to be amplified. PCR can be used to amplify specific RNA sequences and cDNA transcribed from total cellular RNA. See generally Mullis et al., 1987; Erlich, 1989. Thus, amplification of specific nucleic acid sequences by PCR relies upon oligonucleotides or “primers” having conserved nucleotide sequences wherein the conserved sequences are deduced from alignments of related gene or protein sequences, e.g. a sequence comparison of mammalian inhibin-α genes. For example, one primer is prepared which is predicted to anneal to the antisense strand and another primer prepared which is predicted to anneal to the sense strand of a cDNA molecule which encodes inhibin-α.
   To detect the amplified product, the reaction mixture is typically subjected to agarose gel electrophoresis or other convenient separation technique and the relative presence of the marker specific amplified DNA detected. For example, marker amplified DNA may be detected using Southern hybridization with a specific oligonucleotide probe or comparing is electrophoretic mobility with DNA standards of known molecular weight. Isolation, purification and characterization of the amplified DNA may be accomplished by excising or eluting the fragment from the gel (for example, see references Lawn et al., 1981; Goeddel et al., 1980), cloning the amplified product into a cloning site of a suitable vector, such as the pCRII vector (Invitrogen), sequencing the cloned insert and comparing the DNA sequence to the known sequence of the marker. The relative amounts of marker mRNA and cDNA can then be determined.
(iii) Measurement of altered marker protein levels in cell extracts or blood or other suitable biological sample, either qualitatively or quantitatively, for example by immunoassay, utilising immunointeractive molecules such as antibodies directed to a monomeric or heterodimeric subunit or directed to the heterodimeric per se. For example, one may use the PO#12 antibody, which is directed to the αC subunit, to detect αβ dimers, α monomeric subunit and/or αC or αN isoform of the α monomeric subunit.
   In one example, one may seek to detect marker-immunointeractive molecule complex formation. For example, an antibody according to the invention, having a reporter molecule associated therewith, may be utilized in immunoassays. Such immunoassays include but are not limited to radioimmunoassays (RIAs), enzyme-linked immunosorbent assays (ELISAs) and immunochromatographic techniques (ICTs), Western blotting which are well known to those of skill in the art. For example, reference may be made to “Current Protocols in Immunology”, 1994 which discloses a variety of immunoassays which may be used in accordance with the present invention. Immunoassays may include competitive assays. It will be understood that the present invention encompasses qualitative and quantitative immunoassays.
   Suitable immunoassay techniques are described, for example, in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen.
   Two-site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent.
   In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilized first antibody.
   An alternative method involves immobilizing the antigen in the biological sample and then exposing the immobilized antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.
   From the foregoing, it will be appreciated that the reporter molecule associated with the antigen-binding molecule may include the following: —
   (a) direct attachment of the reporter molecule to the antibody;
   (b) indirect attachment of the reporter molecule to the antibody; i.e., attachment of the reporter molecule to another assay reagent which subsequently binds to the antibody; and
   (c) attachment to a subsequent reaction product of the antibody.
   The reporter molecule may be selected from a group including a chromogen, a catalyst, an enzyme, a fluorochrome, a chemiluminescent molecule, a paramagnetic ion, a lanthanide ion such as Europium (Eu³⁴), a radioisotope including other nuclear tags and a direct visual label.
   In the case of a direct visual label, use may be made of a colloidal metallic or non-metallic particle, a dye particle, an enzyme or a substrate, an organic polymer, a latex particle, a liposome, or other vesicle containing a signal producing substance and the like.
   A large number of enzymes suitable for use as reporter molecules is disclosed in U.S. Pat. Nos. 4,366,241, 4,843,000, and 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.
   Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al., International Publication No. WO 93/06121. Reference also may be made to the fluorochromes described in U.S. Pat. Nos. 5,573,909 [Singer et al], 5,326,692 [Brinkley et al]. Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.
   In the case of an enzyme immunoassay, an enzyme is conjugated to the second antibody, generally by means of glutaraldehyde or periodate. As will be readily recognized, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex, allowed to bind, and then the excess reagent washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.
   Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules may also be employed.
(iv) The use of aptamers in screening for nucleic acid molecules or expression products
(v) Determining altered protein expression based on any suitable functional test, enzymatic test or immunological test in addition to those detailed in point (iii)—above.

As detailed above, any suitable technique may be utilised to detect inhibin-α, activin-β_{A}, activin-β_{B}, activin-β_{C}, activin-β_{D}, activin-β_{E} or follistatin or its encoding nucleic acid molecule. The nature of the technique which is selected for use will largely determine the type of biological sample which is required for analysis.

As would be appreciated, depending on the circumstances of a particular patient, it may be necessary to establish whether the patient falls into the category of an “early stage” analysis or an “advanced stage” analysis. This will enable the significance of the results in relation to the levels which are determined for the marker molecules to be placed into the correct context. Establishing the context within which a patient is the subject of analysis can be performed by any suitable means which could be designed and implemented by the person of skill in the art. For example, one may analyse a tissue section, such as a prostate tissue section, in terms of both a morphological analysis of the cell types which are present and the levels of two or more of activin β_{A}, activin β_{C}, inhibin α and/or follistatin protein or expressed gene levels. Alternatively, and more preferably, one may perform a serum analysis to determine the levels of one or more of the subject proteins and consider this result together with the histological findings of a prostate needle biopsy. This would enable one to correlate the presence of a specific grade neoplastic cells with the levels of two or more of activin β_{A}, activin β_{C}, inhibin α and/or follistatin and thereby determine the predisposition of the subject neoplastic cells to progress to a higher grade.

Means of screening for the presence of neoplastic cells in an organ, if necessary, can be achieved by any suitable method which would be well known to the person of skill in the art. For example one may harvest a population of cells for analysis via organ biopsy (for example, a needle biopsy), organ removal or aspiration. Where a section of intact organ is available, one may prepare tissue sections for morphological analysis—such tissue sections may be prepared in any suitable manner such as in the form of frozen sections or wax embedded sections. To the extent that needle biopsies are utilised, one may nevertheless harvest tissue of sufficient size to enable analysis of the tissue's architecture. To the extent that cellular aspirates are harvested, it may be necessary to render the cells a single cell suspension and analyse the morphology of a population of cells derived therefrom. This is a less ideal method but may nevertheless achieve the objective of identifying the existence of particular grade neoplastic cells. In terms of the morphological analysis of these specimens, any suitable histological technique will enable the grading of the cells. For example, tissues can be harvested and stored in the form of formalin fixed tissue, frozen sections, gluteraldehyde fixed or bouins fixed tissue. In terms of histological techniques for achieving morphological analysis, one could utilise, inter alia, haemotoxylin and eosin, immunoperoxidase, electronmicroscopy, in situ staining or PCR in situ.

Reference to a “biological sample” should be understood as a reference to any sample of cells or tissue which is derived from an organism. The cells may be single cells, cultured cells or part of a tissue. In this regard, the biological sample may be derivable from any human or non-human mammal, as detailed above. It should be further understood that reference to “organism” includes reference to embryos and fetuses.

The biological sample may be any sample of material derived from the organism. This includes reference to both samples which are naturally present in the organism, such as tissue and body fluids in a mammal (for example biopsy specimens such as lymphoid specimens, resected tissue, tissue extracts, blood, lymph fluid, feces, bronchial secretions or cell culture medium) and samples which are introduced into the body of the organism and subsequently removed, such as, for example, the saline solution extracted from the lung following a lung lavage or from the colon following an enema. It also includes reference to cells which originated from an organism but have been maintained in vitro, for example cell lines, or which have been manipulated or treated subsequently to removal from the organism, for example immortalised or genetically modified cells or tissues.

The biological sample which is tested according to the method of the present invention may be tested directly or may require some form of treatment prior to testing. For example, a biopsy sample may require homogenisation prior to testing. Where the sample comprises cellular material, it may be necessary to extract or otherwise expose the nucleic acid material present in the cellular material in order to facilitate analysis of the nucleic acid material in terms of its mRNA expression, for example. In yet another example, the sample may be partially purified or otherwise enriched prior to analysis. For example, to the extent that a biological sample comprises a very diverse cell population, it may be desirable to select out a sub-population of particular interest.

The choice of what type of sample is most suitable for testing in accordance with the method disclosed herein will be dependent on the nature of the condition which is being monitored. For example, if the neoplastic condition is a lymphoma, a lymph node biopsy or a blood or marrow sample would likely provide a suitable source of tissue for testing. Consideration would also be required as to whether one is monitoring the original source of the neoplastic cells or whether the presence of metastases or other forms of spreading of the neoplasia from the point of origin is to be monitored. In this regard, it may be desirable to harvest and test a number of different samples from any one organism.

Although the method of the present invention is most conveniently performed by analysis of an isolated biological sample, it should also be understood that reference to analysing a sample “derived from” a mammal includes reference to analysing the sample in vivo.

Another aspect of the present invention provides a diagnostic kit for assaying biological samples comprising an agent for detecting two or more of inhibin-α, activin-β_{A}, activin-β_{B}, activin-β_{C}, activin-β_{D}, activin-β_{E} or follistatin protein or encoding nucleic acid molecule and reagents useful for facilitating the detection by the agent in the first compartment. Further means may also be included, for example, to receive a biological sample. The agent may be any suitable detecting molecule.

As detailed hereinbefore, it has also been surprisingly determined that increased levels of activin-β_{A}, activin-β_{B}, and follistatin, individually, are indicative of the onset or predisposition to the onset of advanced stage cancer. Accordingly, it should be understood that in a related aspect all the previously described embodiments of the present invention which related to the detection and/or monitoring of an onset or predisposition to an onset of an advanced stage cancer should extend to analysis based on screening for changes to the levels of any one of activin-β_{A}, activin-β_{B}, and follistatin.

Further features of the present invention are described in the following non-limiting Examples.

### EXAMPLE 1

### Expression of a Panel of Markers in High Grade Prostate Cancer

Immunohistochemistry was performed on radical prostatectomy tissue from 28 prostate cancer patients with Gleason Grade sum of greater than or equal to 7. After being deparaffinated the tissue underwent a pretreatment step of microwave heating in 0.1M glycine (pH 4.5) for activin-β_{C}, and 0.01M citrate (pH 6) for inhibin-α, follistatin 315 and activin-β_{A}. The sections were immunostained for activin-β_{C} subunit, inhibin-α, follistatin 315 and activin-β_{A} protein using the DAKO Autostainer (DAKO, Carpinteria, USA). Briefly, endogenous peroxidase was blocked by incubation of sections with 0.03% H₂O₂ for 5 minutes (DAKO, Carpinteria, USA). After incubation with CAS Blocking solution (Zymed, CA, USA) for 10 minutes, the sections were incubated with activin-β_{C} clone 1 antibody (working concentration 0.45 μg/ml), inhibin-α PO12 antibody (working concentration 5 μg/ml), follistatin 315 H10 antibody (working concentration 6.7 μg/ml) and activin-β_{A} E4 antibody (working concentration 2.5 μg/ml) for 60 minutes. The antibody was detected by incubation with Envision polymer-antimouse-horse radish peroxidase (DAKO, Carpinteria, USA) for 15 minutes and visualized by reaction with diaminobenzidine (DAB) (DAKO, Carpinteria, USA) for 5 minutes. Activin-β_{C}, activin-β_{A}, follistatin 315, and inhibin-α staining intensity was recorded by histopathologists as no staining=1, variable+/−staining=2, 1+ staining=3, 2+ staining=4, 3+ staining=5.

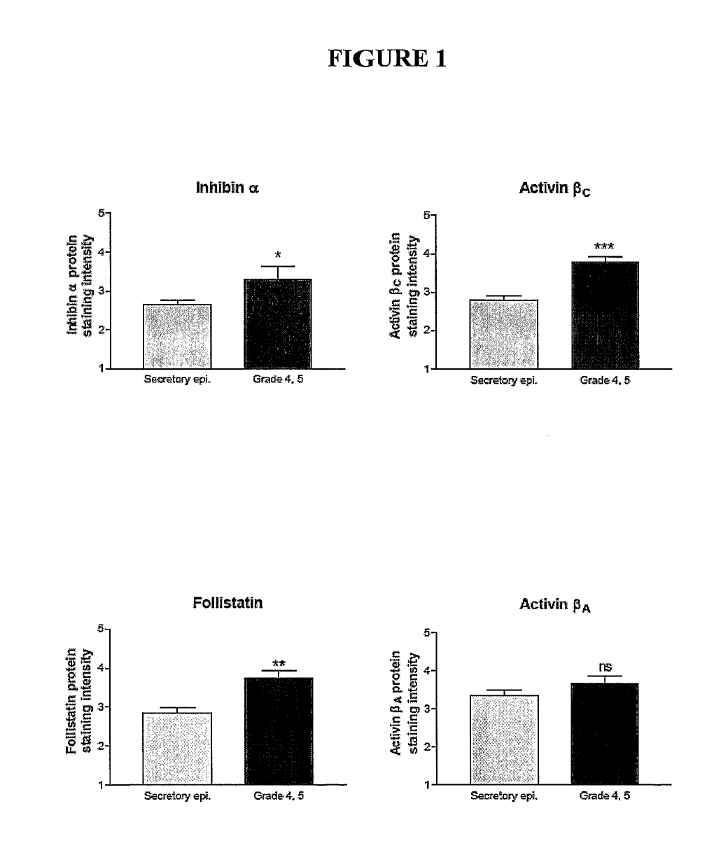

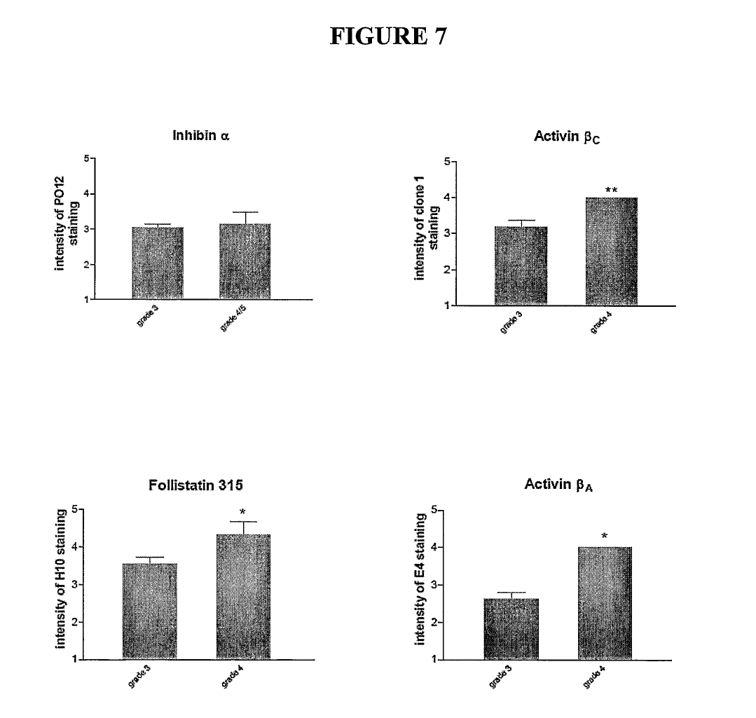

Inhibin-α, activin-β_{C} and follistatin 315 immunostaining was significantly increased in high grade cancers of Gleason grade 4 or 5 compared to adjacent benign secretory epithelium (p<0.05; FIG. 1). In addition, immunostaining for activin-β_{C}, follistatin 315 and activin-β_{A} significantly increased in Gleason grade 4 or 5 prostate cancer compared to Gleason grade 3 prostate cancer (p<0.05; FIG. 7).

These data support the hypothesis that the expression of these four markers is increased during the progression of prostate cancer.

### EXAMPLE 2

### Expression a Panel of Markers in Cribriform Prostate Cancers

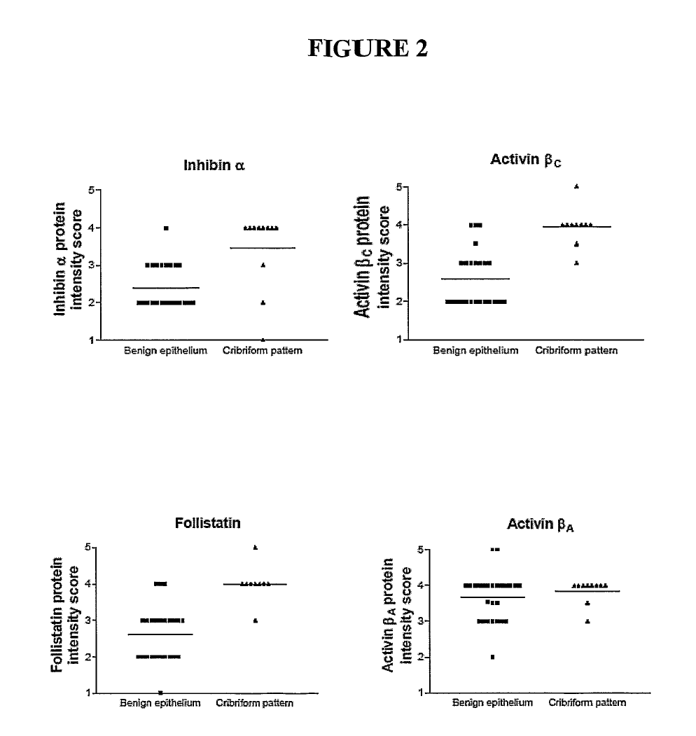

Immunohistochemistry was performed on radical prostatectomy tissue from 28 prostate cancer patients with Gleason Grade sum of greater than or equal to 7. Sections were immunostained as described above. Staining intensity for each of the four markers was recorded by histopathologists as no staining=1, variable+/−staining=2, 1+ staining=3, 2+ staining=4, 3+ staining=5. In FIG. 2, the horizontal bar represents the mean of each immunostaining score in either benign epithelium or cribriform cancer.

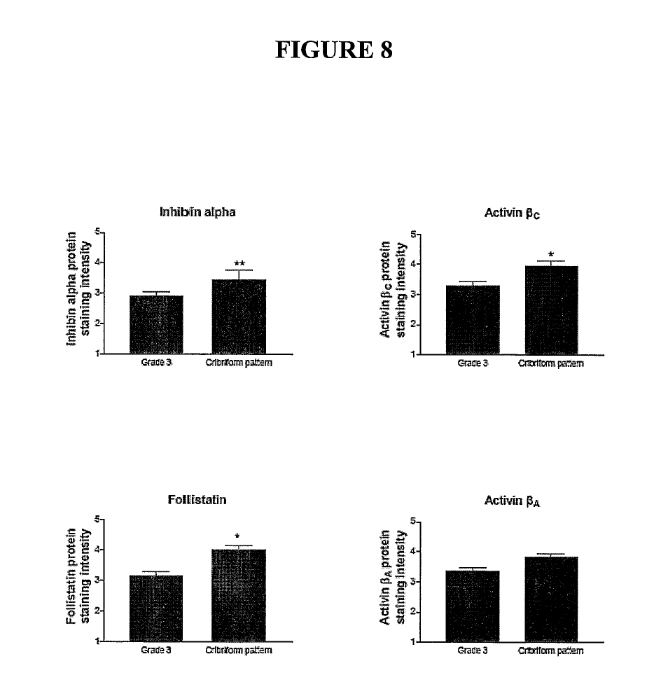

All four markers were expressed in benign secretory epithelium. Inhibin-α, activin-β_{C} and follistatin 315 immunostaining were significantly increased in cribriform cancers compared to benign epithelium (p<0.05; FIG. 2). In addition, immunostaining for inhibin-α, activin-β_{C} and follistatin 315 significantly increased in cribriform pattern prostate cancer compared to Gleason grade 3 prostate cancers (p<0.05; FIG. 8). Activin-β_{A} was not significantly different in cribriform cancers compared to either benign epithelium or Gleason grade 3 cancer (FIGS. 2 and 8). Cribriform cancers are a histopathological sub-type of Gleason grade 3 or 4 prostate cancers based on cellular morphology. Patients diagnosed with cribriform pathology have poor outcome and survival compared to other grade 3/4 prostate cancers [McNeal and Yemoto, 1996, Am J Surg Pathol, 20: 802-14; Rubin et al., Am J Surg Pathol, 22: 840-8, 1998; Wilcox et al., Hum Pathol, 29: 1119-23, 1998; Cohen et al., 2000, Prostate, 43: 11-9]. Therefore, these data support the hypothesis that the expression of the panel of markers is increased during progression of prostate cancers and is associated with poor patient prognosis.

### EXAMPLE 3

### Evaluation of Functional Significance of Activin-β_{C} and Inhibin-α Expression in Non-Aggressive Vs Highly Aggressive Prostate Cancer Cell Lines

Cell Lines

Human prostate tumor epithelial cell lines LNCaP and PC3, were obtained from American Type Culture Collection (Rockville, Md., USA). Cell lines were routinely cultured in Dulbecco's Modified Eagle's Medium (DMEM; Gibco. NY, USA) with 10% (v/v) heat-inactivated foetal calf serum (FCS) (PA Biologicals Co. Pty Ltd, NSW, Australia) and antibiotics (100 UI/ml penicillin and 10 μg/ml streptomycin; CSL Ltd, Parkville, Vic, Australia) in 75 cm² culture flasks (Costar; Corning Costar Corp., Cambridge, Mass., USA) at 37° C. in a humidified atmosphere of 5% CO₂ in air. Cell lines were passaged every four days by trypsinisation.

Transient Transfection of LNCaP and PC3 Cell Lines

Human activin-β_{C} cDNA was subcloned into the pRK5 expression vector. LNCaP cells were plated at a density of 240,000 cells per well, in DMEM+5% FCS into 12-well plates (Falcon) for 48 hrs. LNCaP cells were transiently transfected with pRK5-β_{C} (6 μg) at a ratio of 1:2 DNA (μg) to Lipofectamine PLUS (μl) and 1:1 ratio of DNA (μg) to Lipofectamine reagent (μl) according to the manufacturer's instructions. PC3 cells were plated at 200,000 cells per well in DMEM+10% FCS into 12 well plates (70-80% confluence) for 24 hrs. PC3 cells were transiently transfected with pRK5-β_{C} or pRK5 control (3 μg) using Superfect (Qiagen, Valencia, Calif.), at a ratio of 1:1.7 (μg DNA to μl Superfect reagent) according to manufacturer's instructions.

Stable Transfection of LNCaP and PC3 Cell Lines

The human INHA cDNA subcloned into pcDNA3.1 was obtained from Invitrogen, Carlsbad, Calif., USA. LNCaP cells seeded at 6.24×10⁵ cells/well in a 6-well plate were cultured (50-80% confluence) for 24 hours. Lipofectamine plus (Invitrogen, Carlsbad, Calif., USA) was then used for transfections according to the manufacturer's instructions. Briefly, the cells were transfected with 3.84 μg high quality, linearised plasmid DNA using 4 μl Lipofectamine and 19.2 μl Lipofectamine plus. After 3 hrs, the transfection media was replaced with DMEM+10% FCS, and after 48 hrs, media was replaced with selection media (DMEM supplemented with 10% FCS and 360 μg/ml zeocin). PC3 cells seeded at 4.8×10⁵ cells/well in a 6-well plate were cultured (50-80% confluence) for 24 hours. Superfect (Qiagen Pty Ltd, Clifton Hill, Victoria, Australia) was then used for transfections according to the manufacturer's instructions. Briefly, the cells were transfected with 8.64 μg plasmid DNA using 14.4 μl of Superfect. After 3 hrs, the transfection media was replaced with DMEM+110% FCS, and after 48 hrs, media was replaced with selection media (DMEM supplemented with 10% FCS and 360 μg/ml zeocin). Individual colonies surviving after 2-3 weeks selection were picked and propagated in DMEM supplemented with 10% FCS and 360 μg/ml zeocin. Integration of the plasmids was confirmed by genomic PCR. Expression of inhibin-α from the plasmids was confirmed by RT-PCR and Western blot with R1 monoclonal antibody raised to the mature αC region of the inhibin-α subunit.

Fluorescence-Activated Cell Sorting (FACS) Analysis

About 1×10⁶ cells were trypsinised, washed in ice-cold PBS, and resuspended in 300 μl PBS. The cells were then fixed in ice-cold 100% ethanol and incubated on ice for 30 mins. After three washings with PBS, cells were incubated with the fluorescent dye propidium iodide (50 μg/ml) (Sigma-Aldrich, Castle Hill, NSW, Australia) and RNase A (100 μg/ml) at room temperature for 30 mins. The cells were analysed on a FACStar Plus (details). The excitation wavelength of the laser is 448 nm; fluorescence of more than 630 nm was measured and cell cycle phases were recorded. Percentage of cells in G_{0/1} and S phases was calculated. Each experiment was repeated twice.

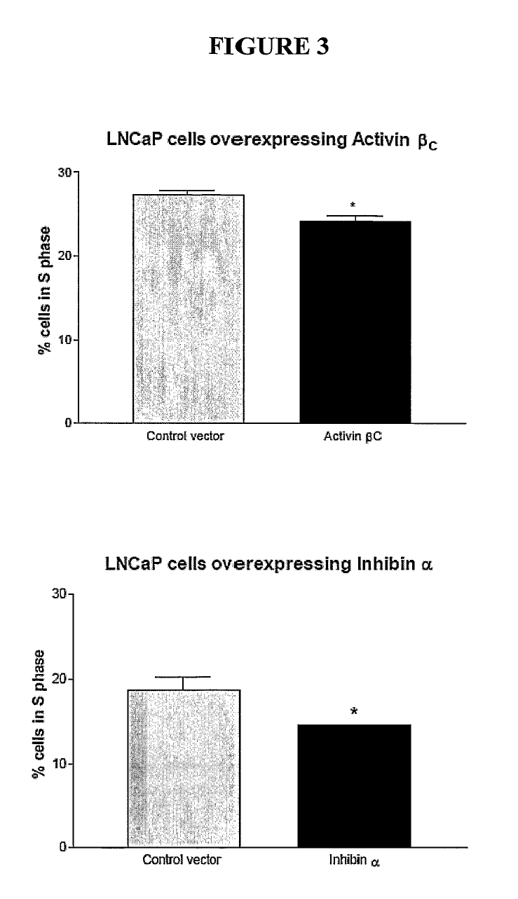

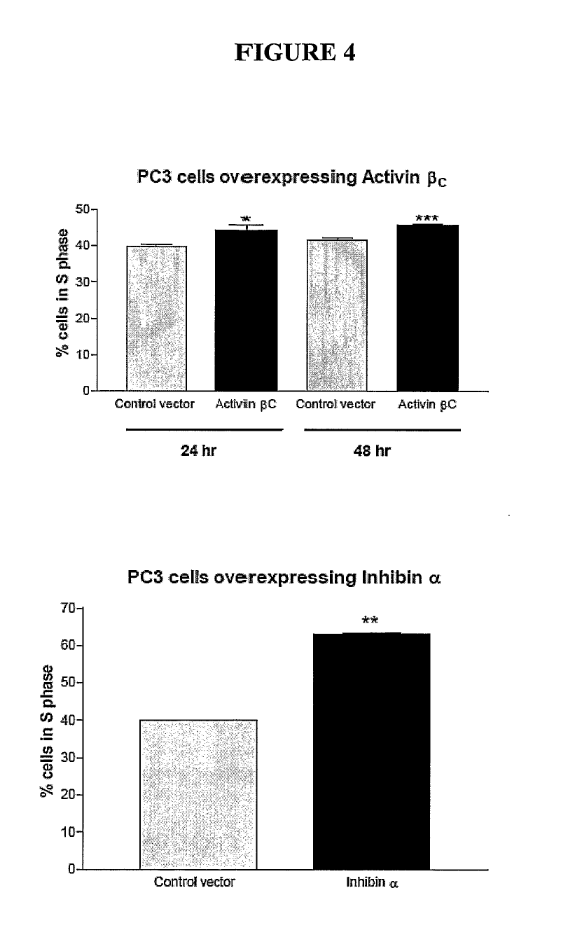

LNCaP cells overexpressing either activin-β_{C} or inhibin-α (black bars) demonstrated a significant reduction in proportion of cells in S-phase compared to LNCaP cells not transfected with activin-β_{C} or inhibin-α cDNA (p<0.05; grey bars; FIG. 3). In contrast, PC3 cells overexpressing either activin-β_{C} or inhibin-α (black bars) demonstrated a significant increase in proportion of cells in S-phase compared to PC3 cells not transfected with activin-β_{C} or inhibin-α cDNA (p<0.05; grey bars; FIG. 4).

LNCaP and PC3 prostate cancer cells represent early and advanced stages of human prostate cancer progression, respectively. LNCaP cells are dependant on androgens and fail to produce tumours in vivo [Fisher et al., Cell Tissue Res, 307: 337-45, 2002]. In contrast, PC3 cells are not dependant on androgens for growth and are highly metastatic in vivo (Fisher et al., 2002, supra). Therefore, these data support the hypothesis that activin-β_{C} and inhibin-α are tumour suppressive in early stage prostate cancer but become growth-promoting or pro-metastatic in advanced prostate cancer.

### EXAMPLE 4

### Expression of a Panel of Markers in Prostate Cancer Metastases to the Lymph Node

Tissue samples from patients with metastatic prostate cancer to the lymph node were obtained from Melbourne Pathology. Activin-β_{A} immunolocalisation was investigated using monoclonal E4 antibody, follistatin immunolocalisation was investigated using the monoclonal H10 antibody and activin-β_{C} was investigated using the monoclonal 25/4 antibody which also detects the activin-β_{E} subunit peptide. Sections were de-paraffinated and underwent antigen retrieval. Sections to be stained were heated in the microwave in antigen retrieval solutions as follows: H10 in 0.1M glycine (pH 4.5), E4 in 0.01 mol/L Tris buffer (pH 9.7) and 25/4 in 0.01M citrate buffer (pH 6). All antibodies were diluted as follows and incubated overnight at 4° C.: E4 antibody was diluted 1:750, H10 was diluted 1:75 and 25/4 antibody was diluted 1:100. The mouse IgG antibody was diluted 1:20 and incubated for 60 minutes at room temperature. Sections were washed with PBS and incubated for 50 min with biotinylated horse antimouse secondary antibody (DAKO Corp., Botany, Australia) at a dilution of 1:200 in PBS. Sections were washed with PBS and incubated with ABC reagent from the Vectastain Elite ABC Kit (Vector Laboratories, Inc., Peterborough, UK) for 40 min. Peroxidase activity was detected using 3,39-diaminobenzidine tetrahydrochloride (Vector Laboratories, Inc.). The reaction was terminated by immersion in distilled water, and the sections were counterstained with Mayers' hematoxylin (Sigma), washed with tap water, dehydrated, and permanently mounted with DPX (BDH, Poole, UK).

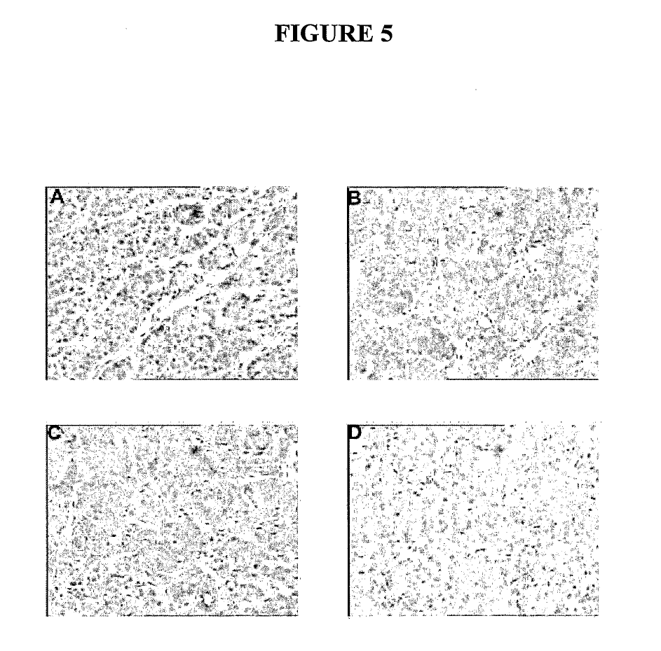

The immunostaining results are shown in FIG. 5. Activin-β_{C} (and/or activin-β_{E})[panel A], follistatin 315 [panel B] and activin-β_{A} [panel C] were all detected immunohistochemically in prostate cancer metastases to the lymph node, while no staining was observed in the negative control [panel D].

### EXAMPLE 5

### Expression a Panel of Markers in Prostate Cancer Metastases to Bone

Metastatic prostate cancer samples were obtained from patients undergoing surgical treatment for bone metastasis. The presence and location of prostate cells within the bone material was confirmed by immunostaining for prostate specific antigen (PSA) on serial sections. Immunostaining in bone metastases was compared to benign secretory epithelium from 18 patients with localised prostate cancer for each of the panel markers. Staining intensity for each of the four markers was recorded by histopathologists as no staining=1, variable+/−staining=2, 1+ staining=3, 2+ staining=4, 3+ staining=5. The horizontal bar represents the mean of each immunostaining score in either benign epithelium or bone metastases.

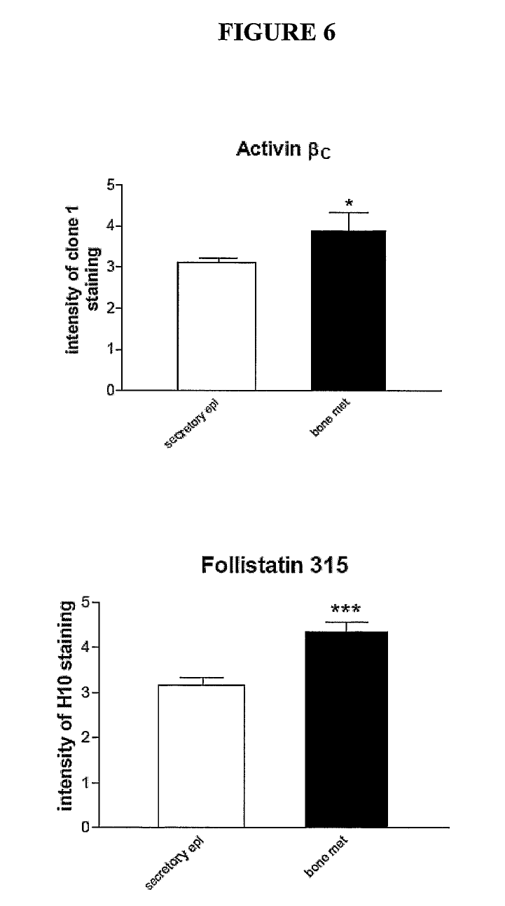

Activin-β_{C} and follistatin 315 were significantly increased in bone metastases compared to benign prostatic secretory epithelium (p<0.05; FIG. 6), supporting the hypothesis that the panel of markers is increased as prostate cancer becomes aggressive.

### EXAMPLE 6

### Modulation of Tumour Growth in vivo

Materials and Methods

Monolayer Wound Healing Assay

The parental LNCaP, empty vector clones (L16, L17, L18) and the inhibin α transfected clones (L1, L5, L8) were plated in triplicates in 6 well plates in DMEM plus 10% FCS and grown until approximately 70-80% confluence. The cell monolayer was then wounded by scraping the surface with a blue 1 ml plastic pipette tip to leave an approximately 1 mm wide clearing. After wounding, the cultures were washed several times with media to remove cells liberated during the wounding process and then cultured in fresh DMEM plus 10% FCS. The same fields were photographed at two or more day intervals using a digital camera. The images were analyzed, wound width measured and plotted as percentage of wound closure relative to day 0. This experiment was repeated twice.

Determination of Tumorigenesis in vivo in SCID Mice

Male SCID mice 6-8 weeks of age were obtained from ARC. They were housed in microisolator cages with free access to sterilized food and water. The experiments were performed a week after the arrival of the animals. All the protocols were approved by the Animal Ethics Committee of Monash Medical Centre, Melbourne, Australia.

The parental LNCaP, empty vector clones (L16, L17, L18) and the inhibin α transfected clones (L1, L5, L8) were used.

A total of 5×10⁶ cells were inoculated subcutaneously in the presence of Matrigel into SCID mice. Control animals were inoculated with Matrigel alone. Mice were maintained in their microisolator cages and were monitored for tumor growth and tumor size weekly until the tumors reached 1 cm when the mice were euthanasized and the tumors excised. Tumor volume (V) was determined using the equation V=(L×W²)×0.5 in which V=volume, L=length, and W=width.

A total of 5×10⁶ cells in 0.01 ml were injected orthotopically into the ventral lobe of the prostate gland after the mice was surgically opened at the lower abdomen. The abdominal wall was sutured using absorbable sutures, and the skin closed with a skin-staple. Mice were weighed twice weekly to ensure close monitoring of health. After 10-12 weeks, the mice were euthanasized and surgically opened for determination of tumor weights.

Results

In vitro wound healing assays can be used to analyze migration. The extent of cell migration was estimated by the rate of the wound closure over a period of time. Wound closure was slower in LNCaP clones overexpressing inhibin α (L1, L5, L8) compared to empty vector clones (L16, L17, L18) and the parental LNCaP cells.

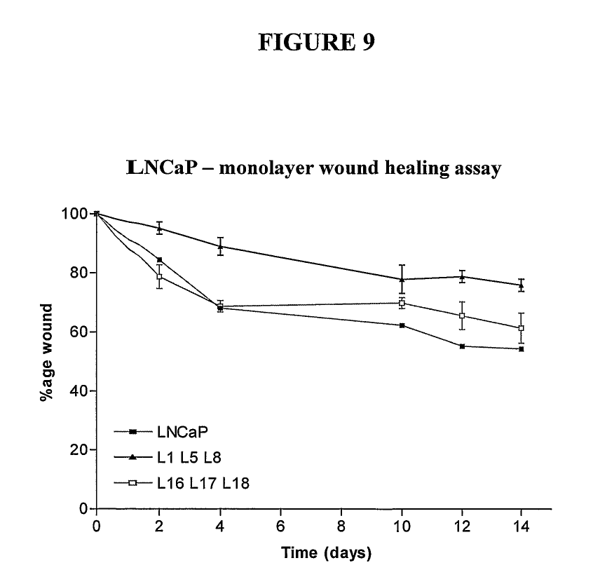

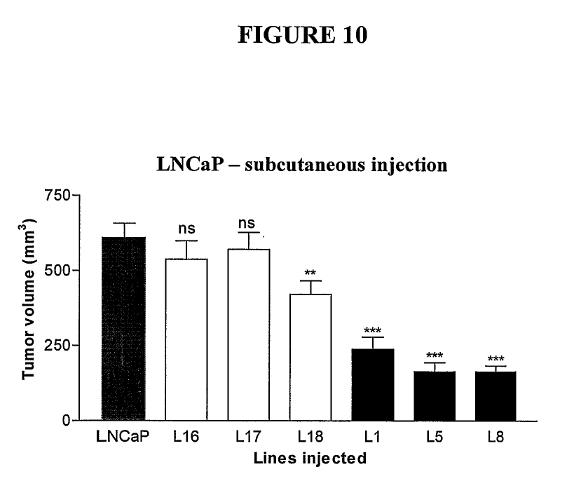

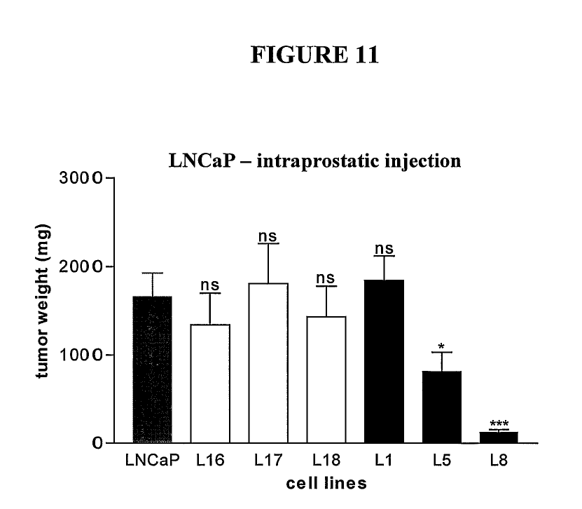

In order to study the effects of inhibin α on tumor growth, an in vivo model of tumor growth in SCID mice was used. When injected subcutaneously, LNCaP clones overexpressing inhibin α demonstrated significant reduction in tumor size (*** p<0.001) compared to the parental LNCaP cells. The prostate tumors formed after orthotopical injection of the LNCaP clones overexpressing inhibin α into the ventral pro state demonstrated significant decrease in tumor weights (* p 0.01-0.05; *** p<0.001) compared to the parental LNCaP cells (FIGS. 9-11).

Overall, the data presented in this study supports the role of inhibin α as a tumor suppressor in prostate cancer.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Balanathan B., Ball E. M. A., Wang H., Harris S. E., Shelling A. N. and Risbridger, G. L., Epigenetic regulation of inhibin α-subunit gene in prostate cancer cell lines, Journal of Molecular Endocrinology (2004) 32, 55-67
Cipriano S. C., Chen, L., Kumar, T. R. and Matzuk, M. M. (2000) ‘Follistatin is a modulator of gonadal tumor progression and the activin- induced wasting syndrome in inhibin-deficient mice’, Endocrinology, 141, 2319-27
Cohen, R. J., McNeal, J. E. and Baillie, T. (2000) Patterns of differentiation and proliferation in intraductal carcinoma of the prostate: significance for cancer progression, Prostate, 43: 11-9.
Fisher, J. L., Schmitt, J. F., Howard, M. L., Mackie, P. S., Choong, P. F. and Risbridger, G. P. (2002) An in vivo model of prostate carcinoma growth and invasion in bone, Cell Tissue Res, 307: 337-45.
Hotten, G., Niedhardt, H., Schneider, C. and Pohl, J. (1995) Cloning of a new member of the TGF-beta superfamily: a putative new activin betaC chain, Biochem Biophys Res Commun, 206: 608-13
Lopez, P., Vidal, F., Rassoulzadegan, M. and Cuzin, F. (1999) ‘A role of inhibin as a tumor suppressor in Sertoli cells: down—regulation upon aging and repression by a viral oncogene’, Oncogene, 18, 7303-9
Matzuk, M. M. and Bradley, A. (1994) ‘Identification and analysis of tumor suppressor genes using transgenic mouse models’, Semin Cancer Biol, 5, 37-45
Matzuk, M. M., Finegold, M. J., Mather, J. P., Krummen, L., Lu, H. and Bradley, A. (1994) ‘Development of cancer cachexia-like syndrome and adrenal tumors in inhibin-deficient mice’, Proc Natl Acad Sci USA, 91, 8817-21
Matzuk, M. M., Finegold, M. J., Su, J. G., Hsueh, A. J. and Bradley, A. (1992) ‘Alpha-inhibin is a tumour-suppressor gene with gonadal specificity in mice’, Nature, 360, 313-9
Matzuk, M. M., Kumar, T. R., Shou, W., Coerver, K. A., Lau, A. L., Behringer, R. R. and Finegold, M. J. (1996) ‘Transgenic models to study the roles of inhibins and activins in reproduction, oncogenesis, and development’, Recent Prog Horm Res, 51, 123-54
McNeal, J. E. and Yemoto, C. E. (1996) Spread of adenocarcinoma within prostatic ducts and acini. Morphologic and clinical correlations, Am J Surg Pathol, 20: 802-14.
Mellor, S. L., Richards, M. G., Pedersen, J. S., Robertson, D. M. and Risbridger, G. P. (1998) ‘Loss of the expression and localization of inhibin alpha-subunit in high grade prostate cancer’, J Clin Endocrinol Metab, 83, 969-975
Moore, A., Basilion, J., Chiocca, E., and Weissleder, R., BBA, 1402: 239-249, 1988.
Pangas, S. A. and Woodruff, T. K. (2000) ‘Activin Signal Transduction Pathways’, Trends Endocrinol Metab, 11, 309-314
Robertson, D. M., Stephenson, T., Cahir, N., Tsigos, A., Pruysers, E., Stanton, P. G., Groome, N. and Thirunavukarasu, P. (2001) Mol Cell Endocrinol, 180: 79-86
Rubin, M. A., de La Taille, A., Bagiella, E., Olsson, C. A. and O'Toole, K. M. (1998) Cribriform carcinoma of the prostate and cribriform prostatic intraepithelial neoplasia: incidence and clinical implications, Am J Surg Pathol, 22: 840-8.
Schmitt, J., Hotten, G., Jenkins, N. A., Gilbert, D. J., Copeland, N. G., Pohl, J. and Schrewe, H. (1996) Structure, chromosomal localization, and expression analysis of the mouse inhibin/activin beta C (Inhbc) gene, Genomics, 32: 358-66
Schmitt, J. F., Millar, D. S., Pedersen, J. S., Clark, S. L., Venter, D. J., Frydenberg, M., Molloy, P. L. and Risbridger, G. P. (2002) ‘Hypermethylation of the inhibin alpha-subunit gene in prostate carcinoma’, Mol Endocrinol, 16, 213-20
Thomas, T. Z., Chapman, S. M., Hong, W., Gurusingfhe, C., Mellor, S. L., Fletcher, R., Pedersen, J. and Risbridger, G. P. (1998) ‘Inhibins, activins, and follistatins: expression of mRNAs and cellular localization in tissues from men with benign prostatic hyperplasia’, Prostate, 34, 34-43
Thomas, T. Z., Wang, H., Niclasen, P., O'Bryan, M. K., Evans, L. W., Groome, N. P., Pedersen, J. and Risbridger, G. P. (1997) ‘Expression and localization of activin subunits and follistatins in tissues from men with high grade prostate cancer’, J Clin Endocrinol Metab, 82, 3851-9
Vale, W., Hseuh, A., Rivier, C. and Yu, J. (1990) In Peptide growth factors and their receptors: Handbook of Experimental Physiology, Vol. 95 (Eds, Sporn, M. and Roberts, A.) Springer-Verlag, Berlin, pp. 211-248
Wedemeyer, N., Potter, T., Wetzlich, S, and Gohde, W. Clinical Chemistry 48: 9 1398-1405, 2002
Weissleder, R., Moore, A., Ph.D., Mahmood-Bhorade, U., Benveniste, H., Chiocca, E. A., Basilion, J. P. Nature Medicine, 6: 351-355, 2000
Wilcox, G., Soh, S., Chakraborty, S., Scardino, P. T. and Wheeler, T. M. (1998) Patterns of high-grade prostatic intraepithelial neoplasia associated with clinically aggressive prostate cancer, Hum Pathol, 29: 1119-23.

1. A method for detecting the onset of a neoplasm or a predisposition to developing a neoplasm in a mammal said method comprising screening for the level of two or more of inhibin-α, activin-β_{A}, activin-β_{B}, activin-β_{C}, activin-β_{D}, activin-β_{E} or follistatin protein and/or gene expression in said mammal wherein a decrease in the level of inhibin-α, activin-β_{A}, activin-β_{B}, activin-β_{C}, activin-β_{D}, activin-β_{E} or follistatin protein and/or gene expression is indicative of the onset of an early stage neoplasm or a predisposition thereto and an increase in the level of inhibin-α, activin-β_{A}, activin-β_{B}, activin-β_{C}, activin-β_{D}, activin-β_{E} or follistatin protein and/or gene expression is indicative of the onset of an advanced stage neoplasm or a predisposition thereto.

2. A method for monitoring for the onset or progression of a neoplasm in a mammal said method comprising screening for the modulation in the level of one or more of inhibin-α, activin-β_{A}, activin-β_{B}, activin-β_{C}, activin-β_{D}, activin-β_{E} or follistatin in said mammal wherein the level of inhibin-α, activin-β_{A}, activin-β_{B}, activin-β_{C}, activin-β_{D}, activin-β_{E} or follistatin relative to the normal level of said inhibin-α, activin-β_{A}, activin-β_{B}, activin-β_{C}, activin-β_{D}, activin-β_{E} or follistatin is indicative of the onset or progression of said neoplasm.

3. The method according to claim 1 or 2 wherein said method is directed to detecting two or more of inhibin-α, activin-β_{A}, activin-β_{C} or follistatin.

4. The method according to claim 3 wherein said method is directed to detecting both inhibin-α and activin-β_{A}.

5. The method according to claim 3 wherein said method is directed to detecting both inhibin-α and activin-β_{C}.

6. The method according to claim 3 wherein said method is directed to detecting both inhibin-α and follistatin.

7. The method according to claim 3 wherein said method is directed to detecting both activin-β_{A} and activin-β_{C}.

8. The method according to claim 3 wherein said method is directed to detecting both activin-β_{A} and follistatin.

9. The method according to claim 3 wherein said method is directed to detecting both activin-β_{C} and follistatin.

10. The method according to claim 3 wherein said method is directed to detecting each of inhibin-α, activin-β_{A} and activin-β_{C}.

11. The method according to claim 3 wherein said method is directed to detecting each of inhibin-α, activin-β_{A} and follistatin.

12. The method according to claim 3 wherein said method is directed to detecting each of inhibin-α, activin-β_{C} and follistatin.

13. The method according to claim 3 wherein said method is directed to detecting each of activin-β_{A}, activin-β_{C} and follistatin.

14. The method according to claim 3 wherein said method is directed to detecting each of inhibin-α, activin-β_{A}, activin-β_{C} and follistatin.

15. The method according to any one of claims 1 to 14 wherein said activin-β_{A}, activin-β_{B}, activin-β_{C}, activin-β_{D}, or activin-β_{E} are in either monomeric or dimeric form.

16. The method according to claim 15 wherein said dimeric form is a homodimer.

17. The method according to claim 15 wherein said dimeric form is a heterodimer.

18. The method according to any one of claims 1 to 14 wherein said inhibin-α is in either monomeric or dimeric form.

19. The method according to claim 18 wherein the form of inhibin-α which is detected is the form of inhibin-α which comprises amino acids 73-96 of the αC region.

20. The method according to any one of claims 3 to 19 wherein said neoplasm is a malignant neoplasm.

21. The method according to claim 20 wherein said malignant neoplasm is a neoplasm of the breast, ovary, thyroid, testis or adrenal gland.

22. The method according to claim 21 wherein said malignant neoplasm is a neoplasm of the breast.

23. The method according to claim 21 wherein said malignant neoplasm is a neoplasm of the ovary.

24. The method according to claim 20 wherein said malignant neoplasm is a neoplasm of the oesophagus, stomach, colon, rectum, kidney, bladder, small intestine, large intestine, larynx, nasal cavity, throat, neural tissue or endometrium or a predisposition to developing an advanced malignant neoplasm of the oesophagus, stomach, colon, rectum, kidney, bladder, small intestine, large intestine, larynx, nasal cavity, throat, neural tissue or endometrium.

25. The method according to claim 20 wherein said malignant neoplasm is a neoplasm of the cervix, brain, skin, lymph node, lung, salivary gland, liver, gallbladder or pancreas.

26. The method according to claim 20 wherein said malignant neoplasm is a neoplasm of the prostate.

27. The method according to any one of claims 21 to 25 wherein said method is directed to detecting a low grade, non-metastatic neoplasm by screening for a decrease in the level of said inhibin-α, activin-β_{A}, activin-β_{C} and/or follistatin.

28. The method according to any one of claims 21 to 25 wherein said method is directed to detecting a high grade neoplasm by screening for an increase in the level of said inhibin-α, activin-β_{A}, activin-β_{C} and/or follistatin.

29. The method according to claim 28 wherein said high grade neoplasm is a metastatic neoplasm.

30. The method according to claim 26 wherein said method is directed to detecting a low grade, non-metastatic prostate neoplasm by screening for a decrease in the level of said inhibin-α, activin-β_{A}, activin-β_{C} and/or follistatin.

31. The method according to claim 26 wherein said method is directed to detecting a high grade prostate neoplasm by screening for an increase in the level of said inhibin-α, activin-β_{A}, activin-β_{C} and/or follistatin.

32. The method according to claim 31 wherein said high grade neoplasm is a metastatic neoplasm.

33. The method according to any one of claims 1 to 32 wherein said screening is performed on a biological sample derived from said mammal.

34. The method according to claim 33 wherein said biological sample is a serum sample.

35. The method according to claim 34 wherein said biological sample is a tissue sample.

36. The method according to claim 26, 30, 31 or 32 wherein said biological sample is a prostate tissue sample.

37. The method according to any one of claims 33 to 36 wherein said screening method is directed to screening for the level of mRNA expression of inhibin-α, activin-β_{A}, activin-β_{B}, activin-β_{C}, activin-β_{D}, activin-β_{E} and/or follistatin.

38. The method according to any one of claims 33 to 36 wherein said screening method is directed to screening for the level of protein expression of inhibin-α, activin-β_{A}, activin-β_{B}, activin-β_{C}, activin-β_{D}, activin-β_{E} and/or follistatin.

39. The method according to claim 38 wherein said inhibin-α protein is detected utilising the PO#12 monoclonal antibody.

40. The method according to any one of claims 1-39 wherein said mammal is a human.

41. A method for detecting the onset of an advanced stage neoplasm or a predisposition to developing a high grade neoplasm in a mammal said method comprising screening for the level of one of activin-β_{A}, activin-β_{B}, or follistatin protein and/or gene expression in said mammal wherein an increase in the level of activin-β_{A}, activin-β_{B} or follistatin protein and/or gene expression is indicative of the onset of a high grade neoplasm or a predisposition thereto.

42. A method for monitoring for the onset or progression of a high grade neoplasm in a mammal said method comprising screening for the modulation in the level of one of activin-β_{A}, activin-β_{B} or follistatin in said mammal wherein the level of activin-β_{A}, activin-β_{B} or follistatin relative to the normal level of said molecule is indicative of the onset or progression of said neoplasm.

43. The method according to any one of claims 41 or 42 wherein said activin-β_{A} or activin-β_{B} are in either monomeric or dimeric form.

44. The method according to claim 43 wherein said dimeric form is a homodimer.

45. The method according to claim 43 wherein said dimeric form is a heterodimer.

46. The method according to any one of claims 41 to 45 wherein said high grade neoplasm is a malignant neoplasm.

47. The method according to claim 46 wherein said malignant neoplasm is a neoplasm of the breast, ovary, thyroid, testis or adrenal gland.

48. The method according to claim 47 wherein said malignant neoplasm is a neoplasm of the breast.

49. The method according to claim 47 wherein said malignant neoplasm is a neoplasm of the ovary.

50. The method according to claim 46 wherein said malignant neoplasm is a neoplasm of the oesophagus, stomach, colon, rectum, kidney, bladder, small intestine, large intestine, larynx, nasal cavity, throat, neural tissue or endometrium or a predisposition to developing an advanced malignant neoplasm of the oesophagus, stomach, colon, rectum, kidney, bladder, small intestine, large intestine, larynx, nasal cavity, throat, neural tissue or endometrium.

51. The method according to claim 46 wherein said malignant neoplasm is a neoplasm of the cervix, brain, skin, lymph node, lung, salivary gland, liver, gallbladder or pancreas.

52. The method according to claim 46 wherein said malignant neoplasm is a neoplasm of the prostate.

53. The method according to any one of claims 46-52 wherein said high grade neoplasm is a metastatic neoplasm.

54. The method according to any one of claims 46 to 53 wherein said screening is performed on a biological sample derived from said mammal.

55. The method according to claim 54 wherein said biological sample is a serum sample.

56. The method according to claim 54 wherein said biological sample is a tissue sample.

57. The method according to claim 52 wherein said biological sample is a prostate tissue sample.

58. The method according to any one of claims 46 to 57 wherein said screening method is directed to screening for the level of mRNA expression of activin-β_{A}, activin-β_{B} or follistatin.

59. The method according to any one of claims 46 to 57 wherein said screening method is directed to screening for the level of protein expression of activin-β_{A}, activin-β_{B} or follistatin.

60. A diagnostic kit for assaying biological samples comprising an agent for detecting two or more of inhibin-α, activin-β_{A}, activin-β_{B}, activin-β_{C}, activin-β_{D}, activin-β_{E} or follistatin protein or encoding nucleic acid molecule and reagents useful for facilitating the detection by the agent in the first compartment when used in the method of any one of claims 1 to 59.
